(19) United States
(12) Patent Application Publication
(10) Pub. No.: US 2008/0027145 A1
(43) Pub. Date: Jan. 31, 2008

(54) Therapeutic Molecules and Methods for Generating and/or Selecting Same

(76) Inventors: David C. S. Huang, North Fitzroy (AU); Peter M. Colman, East Melbourne (AU); Catherine L. Day, Maori Hill (NZ); Jerry M. Adams, North Melbourne (AU); Lin Chen, Rosanna East (AU); Simon N. Willis, Keilor (AU); Andrew Wei, Blackburn (AU); Brian J. Smith, Sunbury (AU); Mark G. Hinds, Brunswick West (AU)

Correspondence Address: THE MCCALLUM LAW FIRM, P. C., 685 BRIGGS STREET, PO BOX 929, ERIE, CO, 80516, US

(21) Appl. No.: 10/588,687

(22) PCT Filed: Feb. 3, 2005
(86) PCT No.: PCT/AU05/00140

§ 371 Date: Jul. 18, 2007

(30) Foreign Application Priority Data
Feb. 6, 2004 (AU) 2004900562

(51) Int. Cl. A61K 35/00; A61P 35/00; G01N 33/48; G01N 33/53

(52) U.S. Cl. 514/789; 436/501; 702/19

(57) ABSTRACT

The present invention relates generally to therapeutic molecules useful for inducing apoptosis of particular cells such as, but not limited to, cancer cells and methods for generating and/or selecting same. The present invention further provides methods for inducing apoptosis of cells such as cancer cells and pharmaceutical compositions useful for same. The present invention further provides methods for generating or selecting therapeutic agents capable of inducing apoptosis of particular cells by the selective inhibition of pro-survival proteins. The present invention further provides a computational approach to therapeutic molecule design based on structure-binding characteristics.

### BACKGROUND OF THE INVENTION

1. Field of the Invention

The present invention relates generally to therapeutic molecules useful for inducing apoptosis of particular cells such as, but not limited to, cancer cells and methods for generating and/or selecting same. The present invention further provides methods for inducing apoptosis of cells such as cancer cells and pharmaceutical compositions useful for same. The present invention further provides methods for generating or selecting therapeutic agents capable of inducing apoptosis of particular cells by the selective inhibition of pro-survival proteins. The present invention further provides a computational approach to therapeutic molecule design based on structure-binding characteristics.

2. Description of the Prior Art

Reference to any prior art in this specification is not, and should not be taken as, an acknowledgment or any form of suggestion that this prior art forms part of the common general knowledge in any country.

Bibliographic details of references provided in this document are listed at the end of the specification.

Cancer is the second leading cause of death in the developed world. Apart from the suffering it causes to patients and their families it is also one of the most expensive diseases to treat (Zhang, Nat Rev Drug Discov 1:101-102, 2002). Accordingly, notwithstanding the toll on human life, if both treatment costs and the cost of reduced economic productivity are considered, the total annual economic burden to society is expected to be in the order of US$200-500 billion by 2010.

Perturbation of programmed cell death (apoptosis) is a central step in the development of many major diseases including cancer. One family of critical regulators of apoptosis is the Bcl-2 protein family. Studies have shown that Bcl-2 overexpression, enforced in human follicular lymphoma, inhibits apoptosis and contributes to tumorigenesis (Vaux et al., Nature 335:440-442, 1988; Strasser et al., Nature 348:331-333, 1990). Bcl-2 overexpression has also been noted in up to 90% of breast, colonic and prostatic cancers (Zhang, 2002, Supra), which represent some of the most common cancers. Pro-survival relatives of Bcl-2 are also overexpressed in many tumors. Indeed, impaired apoptosis is now accepted as a central step in the development of most forms of malignancy (Cory et al., Nat Rev Cancer 2:647-656, 2002).

Impaired apoptosis is also a major impediment to the efficacy of cytotoxic cancer therapy (Cory et al., 2002, Supra; Johnstone et al., Cell 108:153-164, 2002). Most cytotoxic agents, including many chemotherapeutic drugs and radiation, indirectly trigger apoptosis through molecules such as the tumor suppressor p53 (Cory et al., 2002, Supra). In most tumors, however, the p53 pathway is inactivated, preventing the signals to initiate apoptosis. Hence, either loss of p53 function or overexpression of Bcl-2 can provoke chemoresistance, a common cause for treatment failure.

Those members of the Bcl-2 protein family that promote cell survival, including mammalian Bcl-2, Bcl-x_{L}, Bcl-w, Mcl-1 and A1, contain three or four BH (B3cl-2 homology) regions of sequence similarity, and finction until neutralized by their BH3-only relatives. These pro-apoptotic antagonists, which include mammalian Bim, Puma, Bmf, Bad, Bik, Hrk, Bid and Noxa, are related to each other and the wider family only by the short BH3 domain (Huang and Strasser, Cell 103:839-842, 2000). In contrast, Bax and Bak, a sub-group of pro-apoptotic family members, share three BH domains with Bcl-2 and have an essential downstream role, probably in permeabilization of intracellular membranes (Wei et al., Science 292:727-730, 2001).

The BH3-only proteins monitor cellular well-being and damage signals trigger their binding to pro-survival Bcl-2-like proteins, thereby initiating cell death (Cory et al., Oncogene 22:8590-8607, 2003; Huang and Strasser, 2000, Supra). Their differential activation, induced by transcriptional cues (e.g. Bim, Puma, Noxa) or various post-translational mechanisms (e.g. Bim, Bmf, Bad, Bid), imparts some signaling specificity (Puthalakath et al., Cell Death Differ 9:505-512, 2002). Once activated, however, the various BH3-only proteins are generally thought to function similarly by targeting all the pro-survival Bcl-2-like proteins (Adams et al., Genes Dev 17:2481-2495, 2003; Cory et al., Oncogene 22:8590-8607, 2003; Huang and Strasser, 2000, Supra). Their interactions have not, however, been systematically characterized, and the few quantitative studies have been confined to Bcl-x_{L} or Bcl-2 (Letai et al., Cancer Cell 2:183-192, 2002; Petros et al., 2000, Supra; Sattler et al., 1997, Supra). Establishing whether the diverse BH3-only proteins and pro-survival family members interact selectively or promiscuously is important for clarifying how cell death initiates (Adams, 2003, Supra; Cory et al., 2003, Supra; Danial and Korsmeyer, Cell 116:205-219, 2004) and is very pertinent to current efforts to develop compounds that mimic the action of BH3-only proteins as novel anti-cancer agents.

In light of the requirement for less toxic and better targeted anticancer therapies, there is a clear need for the identification of molecules which can interact with Bcl-2-like proteins and inhibit their pro-survival function.

### SUMMARY OF THE INVENTION

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Abbreviations used herein are defined in Table 1.

The present invention provides small molecule antagonists of pro-survival molecules and in particular small molecule antagonists of one or more members of the Bcl-2 family of pro-survival molecules or other related pro-survival molecules. The generation and/or selection of the antagonists is based on mimetics of the natural antagonists of Bcl-2 family proteins (the BH3-only proteins) and/or mimetics of the structural similarities between the Bcl-2 molecules which are only inhibited by a narrow range of BH3-only proteins. Structural studies have revealed that the hydrophobic face of the amphipathic α-helix formed by the BH3 domain of the pro-apoptotic proteins inserts into a hydrophobic groove formed by the BH1, BH2 and BH3 domains of the pro-survival proteins and inhibits their pro-survival functions. The α-helix in the pro-apoptotic proteins comprises hydrophobic regions on the outer sections of the helical terms referred to as H1, H2, H3 and H4. The amino acid sequence forms a heptad repeat. The H1 through H4 outer faces interact with pockets (grooves) on the Bcl-2 proteins.

In accordance with the present invention, the BH3-only proteins are distinguishable functionally with respect to the spectrum of Bcl-2 target molecules to which they interact. These groups are classified in accordance with the present invention as promiscuous and restrictive. By identifying the amino acid charge, size, conformation, solubility, polarity, hydrophobicity, hydrophilicity and contribution to tertiary structure differences between the promiscuous and restrictive BH3-only proteins, especially surrounding the interaction of the α-helix of the BH3 domain and the hydrophobic grooves of the Bcl-2 proteins, mimetics can be generated or selected which mimic one or the other of these groups. The Bcl-2 proteins also have structural features which contribute to the promiscuous or restrictive activity of the BH3-only protein.

For example, amino acids in the heptads repeat on the pro-apoptotic molecule may be modified to reduce an ability for the amino acid or proximal amino acids in the tertiary structure to fit into a pocket formed by the tertiary structure on the Bcl-2 proteins.

Restrictive BH3-only proteins provide, therefore, a scaffold having a conformation conferring a selective ability to antagonize particular Bcl-2 proteins. The scaffold may be used in accordance with the present invention as a template to design mimetics or to model compounds including promiscuous BH3-only proteins to generate antagonists with a restrictive binding spectrum.

Accordingly, in one embodiment of the present invention, mimetics are made to the restrictive BH3-only proteins enabling apoptosis to be induced in selected types of cells such as, but not limited to, cancer cells.

In yet another embodiment of the present invention, the promiscuous and restrictive BH3-only proteins differ in relation to the level of interaction with binding grooves present on the pro-survival Bcl-2 proteins.

The present invention also provides computational methods for predicting the conformation of a molecule which mimics a restrictive BH3-only protein scaffold to generate and/or select and/or screen candidate agents which may then be made and evaluated experimentally for their capacity to induce apoptosis.

The present invention provides in another embodiment, a method for generating or selecting an antagonist of the pro-survival Bcl-2 protein family said method comprising selecting a scaffold BH3-only protein structure with residue positions defining an amphipathic α-helix formed by the BH3 domain; selecting one or more residue positions associated with a promiscuous binding phenotype of a BH3-only protein; substituting amino acid residues conferring a promiscuous phenotype for amino acids or their chemical analogs which confer a restrictive binding pattern to a Bcl-2 protein; and analyzing the interaction of each substitution for an ability to induce a more restrictive spectrum of binding to a Bcl-2 protein.

The present invention further provides a method for generating or selecting an antagonist of the pro-survival Bcl-2 protein family said method comprising selecting a restrictive BH3-only protein as a scaffold protein, determining the conformation of the scaffold conferring the restrictive phenotype and generating or screening for a chemical compound which mimics said scaffold and/or conformational part conferring a restrictive spectrum of binding to a Bcl-2 protein.

The present invention provides, therefore, a computational method for designing an antagonist of the pro-survival Bcl-2 protein family based on a scaffold BH3-only protein with residue positions conferring a restrictive phenotype the method comprising selecting a collection of promiscuous BH3-only proteins; providing a sequence alignment of these proteins and comparing same to a restrictive BH3-only protein; generating a frequency of occurrence for individual amino acids in one or a plurality of positions with said alignments conferring promiscuity or restrictivity with respect to binding to Bcl-2 proteins; creating a scoring fimction selected from charge, size, conformation, solubility, polarity, hydrophobicity, hydrophilicity and contribution to tertiary structure using said frequencies; using said scoring function and/or at least one additional scoring function to generate a set of optimized protein sequences or their conformational equivalents and generating or selecting a compound or protein having a restrictive binding phenotype to a Bcl-2 protein.

In yet another embodiment, the present invention provides the use of a promiscuous BH3-only protein in the generation or selection of amino acid substitution variants which confer a restrictive binding phenotype to said BH3-only protein or its chemical or conformational equivalent.

Still a further aspect of the present invention contemplates a method for generating or selecting an antagonist of a Bcl-2 protein, said method comprising determining a series of parameters selected from:
(1) identifying structural dissimilarities between promiscuous and restrictive Bcl-2 proteins;
(2) identifying structural dissimilarities between promiscuous and restrictive BH3-only proteins; and
(3) identifying structural features of Bcl-2-BH3-only protein complexes and then designing a mimetic of a BH3-only protein which binds to a restrictive range of Bcl-2 proteins.

Mimetics of the BH3-only proteins may also be generated or selected by methods such as, but not limited to, in silico screening, high throughput chemical screening, function-based assays or structure-activity relationships.

In still yet another embodiment, the BH3-only mimetics of the present invention are conveniently provided in medicament form such as a pharmaceutical composition.

The mimetics of the present invention are particularly useful in treating subjects with cancer or a propensity to develop cancer.

### BRIEF DESCRIPTION OF THE FIGURES

FIG. 1 is a graphical representation showing competitive binding assays. (A) Mcl-1 was injected onto sensor chips with mutant ^{4E}BimBH3 (blue) or ^{wt}BimBH3 (red) immobilized. To obtain absolute binding (black), the baseline response with ^{4E}BimBH3 was subtracted from that with ^{wt}BimBH3. (B) Pro-survival proteins bind Bim equally. Sensorgrams showing comparable responses when pro-survival proteins were injected onto immobilized ^{wt}BimBH3. (C) Solution competition assay. Increasing competitor peptide concentration (2) decreases BCl-x_{L} binding to the immobilized ligand (1). (D) Pre-incubation with a competitor BH3 peptide diminishes biosensor responses. Bcl-x_{L} was pre-incubated with increasing concentrations of BikBH3 before the mixture was injected over ^{wt}BimBH3 chip. The line (at 430 s) indicates the response used for calculating the IC50. (E) BikBH3 effectively competed with immobilized BimBH3 for Bcl-x_{L} binding. The relative response (%) indicates the proportion of Bcl-x_{L} that still binds the immobilized peptide in the presence of indicated concentrations of BikBH3, compared to Bcl-x_{L} without BikBH3 (100%). (Irregular responses after 500 s are due to washing the chips after analyte dissociation). Color reproductions are available from the patentees on request.

FIG. 2 is a graphical representation showing pro-apoptotic BH3-only and pro-survival Bcl-2-like proteins have distinctive interactions. (A) Using competitive binding assays, the IC50 (nM) for the indicated interactions were determined. The results shown are from representative experiments; the variation observed in multiple experiments was less than two-fold (using different chips or protein batches). (B) Relative binding affinities of interactions (tabulated in A) were inversely plotted. The BH3 binding profiles of pro-survival proteins are shown. In (C), the sequences of human Bcl-2 (residues 93-202), human Bcl-x_{L} (86-195), human Bcl-w (42-151), mouse Mcl-1 (190-300) and mouse A1 (33-148) that form their BH3 binding grooves (α-helices 2 to 8; Hinds et al., EMBO J 22:1497-1507, 2003) were compared and presented as a phylogenetic tree. Color reproductions are available from the patentees on request.

FIG. 3 is a photographical representation showing Bad, Bik and Noxa have selective pro-survival targets in mammalian cells. Interactions between FLAG (FL)-tagged pro-survival proteins (human Bcl-2, human Bcl-x_{L} and mouse Mcl-1) and HA-tagged BH3-only proteins (A, human Bim; B, human Puma; C, human Bik; D and E, mouse Bad; F and G, mouse Noxa) were tested by co-immunoprecipitation. Equivalent ³⁵S-labeled lysates harvested from 293T cells were immunoprecipitated with antibodies to the HA, FLAG (FL) or control (C) tags. Bim (A) or Puma (B) bound Bcl-2, Bcl-x_{L} and Mcl-1 well. In the top panel of A, Bim_{L} was used instead of Bim_{EL} to discriminate Bim from Bcl-2 by size. (C) Bik preferentially bound Bcl-x_{L}. (D) Bad bound Bcl-2 and Bcl-x_{L} but not Mcl-1, as confirmed by immunoblotting the same filters with the indicated antibodies (E). **Endogenous 14-3-3, associating with Bad, as confirmed by immunoblotting (E). (F) Noxa only bound Mcl-1, as confirmed by immunoblotting (G). *Degradation product of Mcl-1 incapable of binding.

FIG. 4 is a graphical representation showing differential targeting of pro-survival Bcl-2-like proteins by the BH3-only proteins. The affinities of BH3-only peptides for pro-survival proteins (tabulated in FIG. 2) were inversely plotted to allow comparison of the BH3 domains. Color reproductions are available from the patentees on request.

FIG. 5 is a graphical representation showing BH3 peptides have a propensity to be α-helical. (A) CD spectra of BH3 peptides and horse heart myoglobin in 30 mM sodium phosphate (pH 7), showing that the BH3 peptides used were largely unstructured (some have a low % of helicity) whereas the control protein horse heart myoglobin formed an α-helical structure as expected under the buffer condition. Minima (arrowed) at 208 nM and 222 nM are typical of polypeptides that are α-helical. (B) CD spectra of BH3 peptides and myoglobin in 20 mM sodium phosphate (pH 7) supplemented with 30% (v/v) TFE, showing that all BH3 peptides have an α-helical conformation similar to that of horse heart myoglobin in the presence of the helix stabilizing solvent TFE (Nelson and Kallenbach, Biochemistiy 28:5256-5261, 1989). Color reproductions are available from the patentees on request.

FIG. 6 is a graphical representation showing pro-apoptotic Bad and Noxa target selective pro-survival Bcl-2-like proteins. The biosensor response to (A) BadBH3 or (B) NoxaBH3-immobilized chips when recombinant pro-survival Bcl-2-like proteins (Bcl-2 ΔC22, Bcl-x_{L} ΔC24, Bcl-w ΔC29, Mcl-1 ΔN151 ΔC23, A1 ΔC20) or irrelevant proteins (GST and LIF-receptor) was injected. Mcl-1 and A1 had no affinity for BadBH3 whereas Bcl-2, Bcl-x_{L}, Bcl-w bind BadBH3 avidly (A). A complementary pattern was observed with NoxaBH3 (B). Color reproductions are available from the patentees on request.

FIG. 7 is a graphical representation showing that BH3-only proteins that bind selective targets have weak killing activity. (A) The affinities of BH3-only peptides for pro-survival proteins (tabulated in FIG. 3A) were inversely plotted to facilitate comparison of the patterns of BH3 binding. (B) Potent killing of MEFs by Bim and Puma, but not Bmf, Bad, Bik, Hrk or Noxa. Immortalized 3T9 MEFs were infected with retroviruses expressing only GFP (control), or one of the BH3-only proteins and GFP. The viability of the infected (GFP^{+ve}) cells was determined by PI exclusion 24 hours after infection. The histograms represent means±1SD of at least 3 experiments. (C) Interactions between FLAG (FL)-tagged pro-survival proteins (Bcl-x_{L} and Mcl-1) and Bims (or its variants) were tested by co-immunoprecipitation. Equivalent lysates from transfected 293T cells were immunoprecipitated with antibodies to Bim, FLAG (FL) tag or a control irrelevant antigen (C). The filter was probed with a rat monoclonal anti-FLAG antibody. *Degradation product of Mcl-1. (D) Bimₛ variants that have restricted binding to the pro-survival proteins are weak killers. Viability of MEFs infected with retroviruses encoding the indicated proteins (GFP^{+ve}) was analyzed 24 hours after infection. The histograms represent means±1SD of at least 3 experiments. (E) Long-term survival of MEFs infected with BH3 expressing retroviruses. One hundred retrovirally-infected cells were plated and the absolute number of GFP^{+ve} colonies formed after 6 days scored. No colonies were obtained after infection with Bims (†), whereas Bimₛ 4E did not affect long-term viability. Bik, Noxa, Bimₛ BadBH3 or Bimₛ NoxaBH3 had modest effects. The data represent the average number of GFP^{+ve} colonies formed±1SD from at least 3 experiments.

FIG. 8 is a graphical representation showing cooperation between different classes of BH3-only proteins. (A) Based on the binding data, a model proposed to explain the weak killing activity of certain BH3-only proteins that have selective targets. (B) Cooperation between pro-apoptotic BH3-only proteins. MEFs were infected with retroviruses co-expressing a BH3-only protein (Bimₛ, Bik, Noxa or Noxa3E) and GFP, or a BH3-only protein and GFP-tagged-Bimₛ, -Bimₛ BadBH3 or -Bimₛ NoxaBH3. Cell viability was scored 24 hours after infection; data represent means±1SD of at least 3 experiments.

FIG. 9 is a representation showing that a less selective Noxa mutant is a potent killer. (A) Interactions between the α-helical BimBH3 region; the numbering refers to mouse Bim_{L}) with the target groove of Bcl-x_{L} (key residues labeled in black). (B) Alignment of the core BH3 regions of human Bim (Bim_{L}) and human Noxa. Mutated Noxa residues are boxed. (C) Increased binding of Noxa mutants to Bcl-x_{L} and Bcl-w. Wild-type or mutant Noxa peptides were tested in solution competition assays for their capacity to bind Bcl-2, Bcl-x_{L}, Bcl-w or Mcl-1. The histograms show the IC50 (nM) for each interaction. †: IC50 >100 μM. (D) Noxa m3 binds both Bcl-x_{L} and Mcl-1. Interactions between Bcl-x_{L} or Mcl-1, and Bimₛ NoxaBH3 m3 were tested by co-immunoprecipitation. *Mcl-1 degradation product. (E) NoxaBH3 m3 is a potent killer. Survival of MEFs 24 hours after infection with the indicated retroviruses. (F) Dose-dependent killing by NoxaBH3 m3. Viability of MEFs infected with NoxaBH3 or NoxaBH3 m3 sorted for low, medium or high GFP expression; data in (E, F) represent means±1SD of at least 3 experiments.

### DETAILED DESCRIPTION OF THE INVENTION

The present invention contemplates methods for generating mimetics of BH3-only proteins proposed to be useful in inducing apoptosis of selected cells and in particular cancer cells. It is proposed that amino acid charge, size, conformation, solubility, polarity, hydrophobicity, hydrophilicity and contribution to tertiary structure similarities between restrictive BH3-only proteins and their respective target Bcl-2 proteins be exploited to generate mimetics of the BH3-only proteins which induce apoptosis.

Before describing the subject invention in detail it is to be noted that the instant invention is not limited to specific therapeutic components, manufacturing methods, dosage regimens, or the like, as such may vary. It is also to be understood that the terminology used herein is for the purpose of describing particular embodiments only and is not intended to be limiting.

It must also be noted that, as used in the subject specification, the singular forms “a”, “an” and “the” include plural aspects unless the context clearly dictates otherwise. Thus, for example, reference to a “therapeutic agent” includes a single agent, as well as two or more therapeutic agents; reference to a “method” includes a single method, as well as two or more methods; a “residue” includes a single residue, as well as two or more residues, and so forth.

Reference herein to “apoptosis” means a form of cell death in which a programmed sequence of events leads to the elimination of cells.

Accordingly, in one embodiment of the present invention, mimetics are made to the restrictive BH3-only proteins enabling apoptosis to be induced in selected types of cells such as, but not limited to, cancer cells.

Reference herein to “cancer cell” means any cell that exhibits abnormal growth and which tends to proliferate in an uncontrolled way and, in some cases, to metastasize. Cancers contemplated herein include, but are not limited to, ABL1 protooncogene, AIDS Related Cancers, Acoustic Neuroma, Acute Lymphocytic Leukaemia, Acute Myeloid Leukaemia, Adenocystic carcinoma, Adrenocortical Cancer, Agnogenic myeloid metaplasia, Alopecia, Alveolar soft-part sarcoma, Anal cancer, Angiosarcoma, Aplastic Anaemia, Astrocytoma, Ataxia-telangiectasia, Basal Cell Carcinoma (Skin), Bladder Cancer, Bone Cancers, Bowel cancer, Brain Stem Glioma, Brain and CNS Tumors, Breast Cancer, CNS Tumors, Carcinoid Tumors, Cervical Cancer, Childhood Brain Tumors, Childhood Cancer, Childhood Leukaemia, Childhood Soft Tissue Sarcoma, Chondrosarcoma, Choriocarcinoma, Chronic Lymphocytic Leukaemia, Chronic Myeloid Leukaemia, Colorectal Cancers, Cutaneous T-Cell Lymphoma, Dermatofibrosarcoma-protuberans, Desmoplastic-Small-Round-Cell-Tumor, Ductal Carcinoma, Endocrine Cancers, Endometrial Cancer, Ependymoma, Esophageal Cancer, Ewing's Sarcoma, Extra-Hepatic Bile Duct Cancer, Eye Cancer, Eye: Melanoma, Retinoblastoma, Fallopian Tube cancer, Fanconi Anaemia, Fibrosarcoma, Gall Bladder Cancer, Gastric Cancer, Gastrointestinal Cancers, Gastrointestinal-Carcinoid-Tumor, Genitourinary Cancers, Germ Cell Tumors, Gestational-Trophoblastic-Disease, Glioma, Gynaecological Cancers, Haematological Malignancies, Hairy Cell Leukaemia, Head and Neck Cancer, Hepatocellular Cancer, Hereditary Breast Cancer, Histiocytosis, Hodgkin's Disease, Human Papillomavirus, Hydatidiform mole, Hypercalcemia, Hypopharynx Cancer, IntraOcular Melanoma, Islet cell cancer, Kaposi's sarcoma, Kidney Cancer, Langerhan's-Cell-Histiocytosis, Laryngeal Cancer, Leiomyosarcoma, Leukaemia, Li-Fraumeni Syndrome, Lip Cancer, Liposarcoma, Liver Cancer, Lung Cancer, Lymphedema, Lymphoma, Hodgkin's Lymphoma, Non-Hodgkin's Lymphoma, Male Breast Cancer, Malignant-Rhabdoid-Tumor-of-Kidney, Medulloblastoma, Melanoma, Merkel Cell Cancer, Mesothelioma, Metastatic Cancer, Mouth Cancer, Multiple Endocrine Neoplasia, Mycosis Fungoides, Myelodysplastic Syndromes, Myeloma, Myeloproliferative Disorders, Nasal Cancer, Nasopharyngeal Cancer, Nephroblastoma, Neuroblastoma, Neurofibromatosis, Nijmegen Breakage Syndrome, Non-Melanoma Skin Cancer, Non-Small-Cell-Lung-Cancer-(NSCLC), Ocular Cancers, Oesophageal Cancer, Oral cavity Cancer, Oropharynx Cancer, Osteosarcoma, Ostomy Ovarian Cancer, Pancreas Cancer, Paranasal Cancer, Parathyroid Cancer, Parotid Gland Cancer, Penile Cancer, Peripheral-Neuroectodermal-Tumors, Pituitary Cancer, Polycythemia vera, Prostate Cancer, Rare-cancers-and-associated-disorders, Renal Cell Carcinoma, Retinoblastoma, Rhabdomyosarcoma, Rothmund-Thomson Syndrome, Salivary Gland Cancer, Sarcoma, Schwannoma, Sezary syndrome, Skin Cancer, Small Cell Lung Cancer (SCLC), Small Intestine Cancer, Soft Tissue Sarcoma, Spinal Cord Tumors, Squamous-Cell-Carcinoma-(skin), Stomach Cancer, Synovial sarcoma, Testicular Cancer, Thymus Cancer, Thyroid Cancer, Transitional-Cell-Cancer-(bladder), Transitional-Cell-Cancer-(renal-pelvis-/-ureter), Trophoblastic Cancer, Urethral Cancer, Urinary System Cancer, Uroplakins, Uterine sarcoma, Uterus Cancer, Vaginal Cancer, Vulva Cancer, Waldenstrom's-Macroglobulinemia, Wilms' Tumor.

Cancers that are particular targets of the present invention are those which produce an excess amount of a Bcl-2 protein or pro-survival relative and/or a reduced amount of a pro-apoptotic molecule which inhibits a Bcl-2 protein.

In yet another embodiment of the present invention, the BH3-only proteins may be promiscuous or restrictive. Reference herein to “promiscuous” means the protein binds to a number of targets (i.e. binds to all or multiple Bcl-2 proteins). Reference herein to “restrictive” means the protein binds only to specific targets (i.e. binds to only one or a few Bcl-2 proteins). The promiscuous and restrictive BH3-only proteins may differ in relation to the level of interaction with binding grooves present on the pro-survival Bcl-2 proteins.

In accordance with the present invention, the term “target” is used to identify a Bcl-2 protein such as Bcl-2, Bcl-x_{L}, Bcl-w, Mcl and A1 or any other pro-survival molecule comprising three or four Bcl-2 homology (BH) regions.

A “target binder” is used to describe a molecule and or mimetic BH3-only proteins and which inhibit the pro-survival proteins. Naturally occurring target binders include Bim, Puma, Bmf, Bad, Bik, Hrk, Bid and Noxa.

The aim of the present invention is to generate or select highly restrictive and specific mimetics which will act as target binders to inhibitors of apoptosis of particular cells such as cancer cells.

The present invention provides in another embodiment, a method for generating or selecting an antagonist of the pro-survival Bcl-2 protein family said method comprising selecting a scaffold BH3-only protein structure with residue positions defining an amphipathic α-helix formed by the BH3 domain; selecting one or more residue positions associated with a promiscuous binding phenotype of a BH3-only protein; substituting amino acid residues for each of the residues conferring a promiscuous phenotype for an amino acid or its chemical analog which confers a restricted binding phenotype to a Bcl-2 protein; and analyzing the interaction of each substitution for an ability to induce a more restrictive spectrum of binding to a Bcl-2 protein.

Reference herein to a “scaffold protein” means a protein (i.e. BH3 -only protein) for which a library of variants is desired. The scaffold protein is used as input in the protein design calculations, and often is used to facilitate experimental library generation. A scaffold protein may be any protein that has a known structure or for which a structure may be calculated, estimated, modeled or determined experimentally.

The present invention further provides a method for generating or selecting an antagonist of the pro-survival Bcl-2 protein family said method comprising selecting a restrictive BH3-only protein as a scaffold protein, determining the conformation of the scaffold conferring the restrictive phenotype and generating or screening for a chemical compound which mimics said scaffold and/or conformational part conferring a restrictive spectrum of binding to a Bcl-2 protein.

In yet another embodiment, the present invention provides the use of a promiscuous BH3-only protein as an amino acid residue substitute matrix in the generation or selection of substitute variants conferring a restrictive binding phenotype to said BH3-only protein or its chemical or conformational equivalent.

In one example, the molecular basis for selectivity of the Noxa BH3-only protein is described.

Noxa BH3 selectively binds Mcl-1 and A1 and does not bind Bcl-2, Bcl-w or Bcl-x_{L}.

A study of the sequences of the BH3 domain of Noxa reveals that the amino acid immediately before H4 is uniquely a basic amino acid in human Noxa and in the two murine Noxa BH3 domains (Table 3). It is proposed that restoration of an acidic residue at that position, as is more commonly found in other BH3 domain sequences, restores binding to Bcl-2, Bcl-w and Bcl-x_{L}. Assay of a mutant human Noxa BH3 domain in which the relevant lysine amino acid is replaced by glutamic acid shows an IC50 for the mutant peptide of 5.8 μM, i.e. at least 17 fold tighter than the wild-type peptide.

A further unique property of the human Noxa BH3 domain is the presence of an aromatic amino acid, phenylalanine at the H3 position. This is the only occurrence of an amino acid with a branched gamma carbon atom (Table 3) and suggests a requirement for more space in the target Bcl-2 family protein to receive the larger amino acid at this position. When the amino acid sequences of the Bcl-2 family proteins are placed in alignment, it is evident that Mcl-1 and A1 contain smaller amino acids in the receptor site for the H3 amino acid of the BH3 domain. This conclusion is possible by drawing on the published three-dimensional structure of Bcl-x_{L} complexed with the Bim BH3 domain (Liu, X. et al., Immunity 19:341-352, 2003) and using the sequence alignment referred to above. Mutation of human Noxa BH3 from F to I at the H3 position results in an IC50 of 1.1 μM for the mutant peptide, i.e. at least 90 fold tighter than wild type. The double mutant, K to E plus F to I shows the changes to be synergistic with an IC50 of 0.1 μM.

This illustrates how the subject invention enables the conversion of a selective BH3 domain into a promiscuous BH3 domain.

Reference herein to an “agent” should be understood as a reference to any proteinaceous or nonproteinaceous molecule derived from natural, recombinant or synthetic sources. Useful sources include the screening of naturally produced libraries, chemical molecule libraries as well as combinatorial libraries, phage display libraries and in vitro translation-based libraries. Particularly useful sources are the modification of a promiscuous BH3 only protein scaffold to generate a restrictive molecule.

In one embodiment, the agents of the present invention useful for the complete suppression of, or substantial decrease in, the levels or activity of the pro-survival functions of Bcl-2 or a pro-survival relative may be proteinaceous or chemical molecules. All such decreases, inhibitions, reductions and down-regulations of a Bcl-2 family protein pro-survival activity are encompassed by the terms “antagonist” or “antagonism” or “antagonizing”.

In relation to agents which are proteinaceous molecules, such molecules include peptides, polypeptide and proteins. In addition, the terms mutant, part, derivative, homolog, analog or mimetic are meant to encompass various forms of an agent which completely suppresses or substantially decreases the pro-survival functions of Bcl-2 family protein.

The agents may be naturally occurring or artificially generated molecules. The agents may be BH-3 only proteins comprising one or more amino acid substitutions, deletions or additions. Agents may be generated by mutagenesis or other chemical methods or generated recombinantly or synthetically. Alanine scanning is a useful technique for identifying important amino acids (Wells, Methods Enzymol 202:2699-2705, 1991). In this technique, an amino acid residue is replaced by Alanine and its effect on the peptide's activity is determined. Each of the amino acid residues of the agent is analyzed in this manner to determine the important structural and/or charge and/or conformational and/or hydrophobic/hydrophilic regions. Agents are tested for their ability to bind to Bcl-2 and for other qualities such as longevity, binding affinity, dissociation rate, ability to cross membranes or ability to induce apoptosis.

Agents of the present invention may also encompass Bcl-2 binding portions of a full-length BH3-only protein. Portions are at least 1, at least 10, least 20 and at least 30 contiguous amino acids, such as 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30 amino acids which define a Bcl-2 binding fragment such as an amphipathic α-helix structure. It is proposed that this structure interacts with the hydrophobic grooves of the Bcl-2 proteins. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of an amino acid sequence of the invention with proteinases such as endoLys-C, endoArg-C, endoGlu-C and staphylococcus V8-protease. The digested fragments can be purified by, for example, high performance liquid chromatographic (HPLC) techniques. Any such fragment, irrespective of its means of generation, is to be understood as being encompassed by the term “antagonist” as used herein. Thus antagonists may comprise a derivative of a promiscuous BH3-only protein. Such a derivative includes parts, mutants, homologs, fragments, analogues as well as hybrid or fusion molecules and glycosylation variants of a promiscuous BH3-only protein. Derivatives also include molecules having a percent amino acid sequence identity over a window of comparison after optimal alignment. Preferably, the percentage similarity between a particular sequence and a reference sequence is at least about 60% or at least about 70% or at least about 80% or at least about 90% or at least about 95% or above such as at least about 96%, 97%, 98%, 99% or greater. Preferably, the percentage similarity between species, functional or structural homologs of the instant agents is at least about 60% or at least about 70% or at least about 80% or at least about 90% or at least about 95% or above such as at least about 96%, 97%, 98%, 99% or greater. Percentage similarities or identities between 60% and 100% are also contemplated such as 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

Analogs of residues in a protein antagonist such as a derivative of a BH3-only protein contemplated herein include but are not limited to modification to side chains, incorporating unnatural amino acids and/or their derivatives during peptide, polypeptide or protein synthesis and the use of crosslinkers and other methods which impose conformational constraints on the proteinaceous molecule or their analogs. This term also does not exclude modifications of the polypeptide, for example, glycosylations, acetylations, phosphorylations and the like. Included within the definition are, for example, polypeptides containing one or more analogs of an amino acid (including, for example, unnatural amino acids such as those given in Table 1) or polypeptides with substituted linkages. Such polypeptides may need to be able to enter the cell.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via 0-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy -5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids, contemplated herein is shown in Table 1. Such unnatural amino acids may be useful in conferring a tertiary structure analogous to a restrictive BH3 only protein scaffold.

TABLE 1
CODES FOR NON-CONVENTIONAL AMINO ACIDS

| | | Non-conventional | |
|---|---|---|---|
| Non-conventional amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Reference to a mimetic of a BH3-only protein includes a target binder (i.e. a BH3-only protein) at the structural and/or functional level and inhibits a pro-survival Bcl-2-protein. In accordance with one embodiment of the present invention, it is proposed to generate selected BH3-only protein mimetics. A BH3-only protein mimetic is designed based on structural differences between targets and structural differences between target binders. The latter may, in accordance with the present invention and as defined hereinbefore, be divided into promiscuous (i.e. binds to all or multiple Bcl-2 proteins) or restrictive (i.e. binds to one or only a few Bcl-2 proteins).

A peptide mimetic may be a peptide-containing molecule that mimics elements of protein secondary structure (Johnson et al., Peptide Turn Mimetics in Biotechnology and Pharmacy, Pezzuto et al., Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to the natural molecule. Peptide or non-peptide mimetics of a BH3-only protein may be useful in the present invention as an agent which decreases the pro-survival function of Bcl-2.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. As described hereinbefore, Alanine scans of peptides are commonly used to refine such peptide motifs. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic. Modelling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design in accordance with the present invention is to use computational methods to generate and/or select structural analogs of restrictive BH3-only proteins in order to fashion drugs which are, for example, more active or stable forms of the polypeptide and which have a restrictive binding spectrum. In one approach, one first determines the three-dimensional structure of a protein of interest by x-ray crystallography, by computer modelling or most typically, by a combination of approaches. Useful information regarding the structure of a polypeptide may also be gained by modelling based on the structure of homologous proteins. An example of rational drug design is the development of HIV protease inhibitors (Erickson et al., Science 249:527-533, 1990).

One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may measure, for example, the formation of complexes between a target or fragment and the agent being tested, or examine the degree to which the formation of a complex between a target or fragment and a known ligand is aided or interfered with by the agent being tested.

The screening procedure includes assaying (i) for the presence of a complex between the drug and the target, or (ii) an alteration in the expression levels of nucleic acid molecules encoding the target. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the compound rather than the target and to measure the amount of compound binding to target in the presence and in the absence of the drug being tested.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for target antagonists or agonists.

Purified target can be coated directly onto plates for use in the aforementioned drug screening techniques. However, non-neutralizing antibodies to the target may also be used to immobilize the target on the solid phase. The target may alternatively be expressed as a fusion protein with a tag conveniently chosen to facilitate binding and identification.

In another embodiment, high throughput chemical screening (HTCS) for inhibitors of Bcl-2 and Bcl-w can be carried out. Given the interaction of a BH3-only protein like Bim with a pro-survival molecule (Bcl-2 or Bcl-w) precipitates apoptosis, libraries can be screened for small organic molecules that bind to the pro-survival proteins in such a way as to prevent BH3 binding. Multiple screening campaigns can be undertaken in order to identify compounds that target one or both anti-apoptotic molecules.

The proteins necessary for the high capacity assays may be produced in bacteria and initial studies using an optical biosensor (BiaCore) show that a biotinylated Bim BH3 peptide binds His₆-tagged Bcl-w ΔC10 with high affinity (K_{d}˜11 nM) (Hinds et al., EMBO J 22:1497-1507, 2003). The high capacity binding assays necessary for HTCS have been developed using AlphaScreen™ (Amplified Luminescent Proximity Homogeneous Assay) technology (Glickman et al., J Biomol Screen 7:3-10, 2002). By revealing changes in fluorescence output as two partner proteins interact, it can monitor protein interactions with exquisite sensitivity. AlphaScreen™ is well suited for HTCS, as it is robust and can readily be carried out in small volumes as a homogenous assay with great dynamic range.

In one embodiment His₆ Bcl-w ΔC10 is bound to nickel-coated acceptor beads and the biotinylated BimBH3 peptide is bound to the streptavidin-coated donor beads. The beads are then incubated with the test compounds in the wells of a 384-well microtitre plate (one test compound per well) and the assay results read using the Fusion alpha plate reader. The binding assay may be optimized with respect to the concentration of the protein partners and beads, incubation times and assay volumes so that the assay typically yields a signal to background ratio of >30:1. The assay has been validated as the IC₅₀ values obtained for a series of peptides were comparable with those obtained using an optical biosensor. Although the affinities of the peptides spanned over 3-orders of magnitude (8 nM-35 μM), the strong correlation observed between the two sets of results (R²=0.9983) indicates that the assays measure the same interactions. The binding assays for His₆ Bcl-2 ΔC22/Bim BH3 may also be optimized. Once the assay is optimised, it could be subjected to a rigorous quality control to assess plate-to-plate and day-to-day reproducibility. Each assay could then be used to screen a unique discovery library. To eliminate false positives, all inhibitory compounds that meet the target potency (IC50 <25 μM) may be validated in secondary competition assays (AlphaScreen™, fluorescence polarisation and BiaCore optical biosensor). The optical biosensor facilitates to quantify the interactions between Bcl-2 family members, and ready comparison between the affinities of strong candidates to the physiological binding by BH3-only proteins can be made.

Compounds that pass these initial tests may be checked for identity and purity by, inter alia, liquid chromatography-mass spectrometry and then tested for their target specificity, i.e. affinity for Bcl-2, Bcl-x_{L}, Bcl-w. Active compounds will also be tested in assays designed to predict intestinal absorption (Wohnsland et al., J Med Chem 44:923-930, 2001) and hepatotoxicity. In addition, in silico methods may be used to predict their bio-distribution properties, and to exclude pharmacophores that could present metabolic or toxicity problems (Drug Metabolism Databases and High-Throughput Testing During Drug Design and Development, Ed Erhardt, Blackwell Science, Malden, Mass., USA, 1999). The data on all the active compounds may be ranked by potency in binding assays, target selectivity, favourable predictive ADMET (Adsorption, Distribution, Metabolism, Excretion and Toxicity) properties (van de Waterbeemd and Gifford, Nat Rev Drug Disc 2:192-204, 2003) and chemical tractability. Then, all available close structural analogues of the top compounds may be obtained and tested for inhibitory activity in binding and killing assays to determine preliminary structure-activity relationships for each structural series.

In respect of assays on lead compounds for biological activity, when promising leads are found, their activity on cell viability in culture may be assessed. Up to 50 lead compounds, optimised according to the criteria described above, may be tested on a panel of cultured Tumorigenic and non-Tumorigenic cell lines, as well as primary mouse and human cell populations, e.g. lymphocytes. Cell viability may be monitored over 3-7 days of incubation with 1 nM-100 μM of the compounds. Greatest attention will, of course, be given to compounds that kill Tumor cells much more efficiently than their normal cell counterparts. Compounds that kill at <10 μM may be evaluated for the specificity of their targets and mode of action. Verifying their mode of action is important, because a test compound might well kill cells indirectly. For example, if a lead compound binds with high selectivity to Bcl-2, it should not kill cells lacking Bcl-2. Hence, the specificity of action may be confirmed by comparing the activity of the compound in wild-type cells with those lacking Bcl-2.

The most promising candidates may be subjected to a thorough analysis of their anti-Tumor efficacy in mouse models. In two models that have fully characterised previously, immuno-competent mice injected with B-cell lymphomas, derived from either myc transgenic mice (Adams et al., Nature 318:533-538, 1985) or myc/bcl-2 doubly transgenic animals (Strasser et al., Supra), succumb rapidly and reproducibly to a leukaemia/lymphoma syndrome. Although both tumors respond to standard chemotherapy (cyclophosphamide), mice injected with myc/bcl-2 Tumor cells invariably relapse. These two transplantable Tumors will allow testing of any compounds, given alone or in combination with cyclophosphamide, in treating these malignancies which closely model human lymphomas.

In respect of structure-activity relationships (SAR) of the lead compounds and their optimisation, the leads selected from initial screens may require considerable modification to enhance their biochemical, biological and pharmacological properties (Bleicher et al., Nat Rev Drug Discov 2:369-378, 2003). To aid optimisation of these compounds, their mode of action may be verified in biochemical and structural studies. Furthermore, complexes formed between the agents and the pro-survival molecules may be analysed by NMR spectroscopy. Because NMR can detect ligands of low affinity and reveal where on the target protein they bind, it can greatly aid the optimisation of binding and accelerate the drug discovery process (Hajduk et al., J Med Chem 42:2315-2317, 1999; Pellecchia et al., Nat Rev Drug Discov 1:211-219, 2002). Using techniques such as chemical shift mapping, binding of test compounds to Bcl-2 proteins will be monitored and those mimicking a BH3 domain will be selected for optimisation.

In a related approach, molecular modelling of the lead agents may be performed to assess their binding in silico using an adapted DOCK program (Kuntz, Science 257:1078-1082, 1992). Lead compounds will be modelled onto the target Bcl-2 groove and scoring functions used to predict the most likely binding modes. This will guide the design of derivatives that provide additional interactions to enhance binding. The availability of NMR-derived experimental data also makes it possible to dock the ligand and the target flexibly in order to predict improved ligands (Lugovskoy et al., J Am Chem Soc 124:1234-1240, 2002).

This information and those from biological assays may be used to synthesise derivative compounds for further testing. For each class of lead compound, a strategy for synthesising derivatives. For example, a typical hit compound is composed of two or three linked ring systems, each of which may be substituted by a range of functional groups. By systematically replacing each of the functional groups, compounds with a wide range of chemical properties can be made and tested.

The present invention also provides computational methods for predicting the conformation of a molecule which mimics a restrictive BH3-only protein scaffold to generate and/or select and/or screen candidate agents which may then be made and evaluated experimentally for their capacity to induce apoptosis.

The present invention provides, therefore, a computational method for designing an antagonist of the pro-survival Bcl-2 protein family based on a scaffold BH3-only protein with residue positions conferring a restrictive phenotype the method comprising selecting a collection of promiscuous BH3-only proteins; providing a sequence alignment of these proteins and comparing same to a restrictive BH3-only protein; generating a frequency of occurrence for individual amino acids in one or a plurality of positions with said alignments conferring promiscuity or restrictivity with respect to binding to Bcl-2 proteins; creating a scoring function selected from charge, size, conformation, solubility, polarity, hydrophobicity, hydrophilicity and contribution to tertiary structure using said frequencies; using said scoring finction and at least one additional scoring function to generate a set of optimized protein sequences or their conformational equivalents and generating or selecting a compound or protein having a restrictive binding phenotype to a Bcl-2 protein.

An assessment of the ability of a restrictive BH3-only protein to antagonize a Bcl-2 protein and induce apoptosis is important for selection of an appropriate therapeutic protocol. Such an assessment is suitably facilitated with the assistance of a computer programmed with software, which inter alia adds a scoring function (SF) for at least one feature associated with the restrictive BH3-only protein to provide a potency value (P_{A}) corresponding to the degree of Bcl-2 antagonism induced. The SF can be selected from, inter alia, (a) the number and position of acidic residues; or (b) the number and position of basic residues; or (c) the number and position of polar residues; or (d) the number and position of non-polar residues; or (e) the number and position of charged residues; or (f) the number and position of uncharged residues; or (g) the number and position of hydrophillic residues; or (h) the number and position of hydrophobic residues; or (i) the levels of residues; or (j) the solubility levels of residues; or (k) the size of residues; or (l) the contribution to tertiary structure the residue makes in the BH3-only protein. Thus, in accordance with the present invention, SF for such features are stored in a machine-readable storage medium, which is capable of processing the data to provide a P_{A} for a particular restrictive BH3-only protein or chemical equivalent.

Thus, in another aspect, the invention contemplates a computer program product for determining the structure of an agent to induce apoptosis in a cell, said product comprising:
(1) code that receives as input scoring finction (SF) for at least two features associated with said BH3-only or Bcl-2 proteins, wherein said features are selected from, inter alia,:
   (a) the number and position of acidic residues;
   (b) the number and position of basic residues;
   (c) the number and position of polar residues;
   (d) the number and position of non-polar residues;
   (e) the number and position of charged residues;
   (f) the number and position of uncharged residues;
   (g) the number and position of hydrophillic residues;
   (h) the number and position of hydrophobic residues;
   (i) the levels of residues;
   (j) the solubility levels of residues;
   (k) the size of residues;
   (l) the contribution to tertiary structure the residue makes in the BH3-only protein
(2) code that adds said SF to provide a sum corresponding to a P_{V} for BH3 -only proteins; and
(3) a computer readable medium that stores the codes.

In a related aspect, the invention extends to a computer for assessing the likely usefulness of a BH3-only protein or chemical equivalent to induce apoptosis in a cell, wherein said computer comprises:
(1) a machine-readable data storage medium comprising a data storage material encoded with machine-readable data, wherein said machine-readable data comprise I_{V}s for at least two features associated with said BH3-only or Bcl-2 proteins, wherein said features are selected from, inter alia,:
   (a) the number and position of acidic residues;
   (b) the number and position of basic residues;
   (c) the number and position of polar residues;
   (d) the number and position of non-polar residues;
   (e) the number and position of charged residues;
   (f) the number and position of uncharged residues;
   (g) the number and position of hydrophillic residues;
   (h) the number and position of hydrophobic residues;
   (i) the levels of residues;
   (j) the solubility levels of residues;
   (k) the size of residues;
   (l) the contribution to tertiary structure the residue makes in the BH3-only protein
(2) a working memory for storing instructions for processing said machine-readable data;
(3) a central-processing unit coupled to said working memory and to said machine-readable data storage medium, for processing said machine readable data to provide a sum of said SF corresponding to a P_{V} for said compound(s); and
(4) an output hardware coupled to said central processing unit, for receiving said P_{V}.

Any general or special purpose computer system is contemplated by the present invention and includes a processor in electrical communication with both a memory and at least one input/output device, such as a terminal. Such a system may include, but is not limited, to personal computers, workstations or mainframes. The processor may be a general purpose processor or microprocessor or a specialized processor executing programs located in RAM memory. The programs may be placed in RAM from a storage device, such as a disk or pre-programmed ROM memory. The RAM memory in one embodiment is used both for data storage and program execution. The computer system also embraces systems where the processor and memory reside in different physical entities but which are in electrical communication by means of a network.

Agents identified in accordance with the present invention are useful in the treatment of cancer.

Reference herein to “treatment” may mean a reduction in the severity of an existing condition. The term “treatment” is also taken to encompass “prophylactic treatment” to prevent the onset of a condition. The term “treatment” does not necessarily imply that a subject is treated until total recovery. Similarly, “prophylactic treatment” does not necessarily mean that the subject will not eventually contract a condition.

Subject as used herein refers to humans and non-human primates (e.g. gorilla, macaque, marmoset), livestock animals (e.g. sheep, cow, horse, donkey, pig), companion animals (e.g. dog, cat), laboratory test animals (e.g. mouse, rabbit, rat, guinea pig, hamster), captive wild animals (e.g. fox, deer), reptiles or amphibians (e.g. cane toad), fish (e.g. zebrafish) and any other organisms (e.g. c. elegans) who can benefit from the agents of the present invention. There is no limitation on the type of animal that could benefit from the presently described agents. The most preferred subject of the present invention is a human. A subject regardless of whether it is a human or non-human organism may be referred to as a patient, individual, animal, host or recipient.

Accordingly, another aspect of the present invention provides a method of preventing or reducing cancer in a subject said method comprising administering to said subject an effective amount of an antagonist of a Bcl-2 protein for a time and under conditions sufficient to prevent or decrease cancer.

The identification of agents, capable of antagonizing Bcl-2 and inducing apoptosis provides pharmaceutical compositions for use in the therapeutic treatment of cancer.

The agents of the present invention can be combined with one or more pharmaceutically acceptable carriers and/or diluents to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., Remington's Pharmaceutical Sciences, 18^{th} Edition, Mack Publishing Company, Easton, Pa., 1990 (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the modulatory agent of the invention and on its particular physio-chemical characteristics.

Administration of the agent, in the form of a pharmaceutical composition, may be performed by any convenient means known to one skilled in the art. Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, patch and implant.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier, see, e.g, International Patent Publication Number WO 96/11698.

Agents of the present invention, when administered orally, may be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g. Fix, Pharm Res 13:1760-1764, 1996; Samanen et al., J Pharm Pharmacol 48:119-135, 1996; U.S. Pat. No. 5,391,377, describing lipid compositions for oral delivery of therapeutic agents (liposomal delivery is discussed in further detail, infra). The pharmaceutical forms suitable for injectable use include sterile aqueous solutions (where water-soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the agents in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

For parenteral administration, the agent may dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the agents are being administered intrathecally, they may also be dissolved in cerebrospinal fluid.

For transmucosal or transdermal administration, penetrants appropriate to the barrier to be permeated can be used for delivering the agent. Such penetrants are generally known in the art e.g. for transmucosal administration, bile salts and fusidic acid derivatives. In addition, detergents can be used to facilitate permeation. Transmucosal administration can be through nasal sprays or using suppositories e.g. Sayani and Chien, Crit Rev Ther Drug Carrier Syst 13:85-184, 1996. For topical, transdermal administration, the agents are formulated into ointments, creams, salves, powders and gels. Transdermal delivery systems can also include patches.

For inhalation, the agents of the invention can be delivered using any system known in the art, including dry powder aerosols, liquids delivery systems, air jet nebulizers, propellant systems, and the like, see, e.g., Patton, Nat Biotech 16:141-143, 1998; product and inhalation delivery systems for polypeptide macromolecules by, e.g., Dura Pharmaceuticals (San Diego, Calif.), Aradigm (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, for example, air jet nebulizers.

The agents of the invention can also be administered in sustained delivery or sustained release mechanisms, which can deliver the formulation internally. For example, biodegradable microspheres or capsules or other biodegradable polymer configurations capable of sustained delivery of a peptide can be included in the formulations of the invention (e.g. Putney and Burke, Nat Biotech 16:153-157, 1998).

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's.

In one aspect, the pharmaceutical formulations comprising agents of the present invention are incorporated in lipid monolayers or bilayers such as liposomes, see, e.g., U.S. Pat. Nos. 6,110,490; 6,096,716; 5,283,185 and 5,279,833. The invention also provides formulations in which water-soluble modulatory agents of the invention have been attached to the surface of the monolayer or bilayer. For example, peptides can be attached to hydrazide-PEG-(distearoylphosphatidyl) ethanolamine-containing liposomes (e.g. Zalipsky et al., Bioconjug Chem 6:705-708, 1995). Liposomes or any form of lipid membrane, such as planar lipid membranes or the cell membrane of an intact cell e.g. a red blood cell, can be used. Liposomal formulations can be by any means, including administration intravenously, transdemially (Vutla et al., J Pharm Sci 85:5-8, 1996), transmucosally, or orally. The invention also provides pharmaceutical preparations in which the nucleic acid, peptides and/or polypeptides of the invention are incorporated within micelles and/or liposomes (Suntres and Shek, J Pharm Pharmacol 46:23-28, 1994; Woodle et al., Pharm Res 9:260-265, 1992). Liposomes and liposomal formulations can be prepared according to standard methods and are also well known in the art see, e.g., Remington's; Akimaru et al., Cytokines Mol Ther 1:197-210, 1995; Alving et al., Immunol Rev 145:5-31, 1995; Szoka and Papahadjopoulos, Ann Rev Biophys Bioeng 9:467-508, 1980, U.S. Pat. Nos. 4, 235,871, 4,501,728 and 4,837,028.

The pharmaceutical compositions of the invention can be administered in a variety of unit dosage forms depending upon the method of administration. Dosages for typical pharmaceutical compositions are well known to those of skill in the art. Such dosages are typically advisorial in nature and are adjusted depending on the particular therapeutic context, patient tolerance, etc. The amount of agent adequate to accomplish this is defined as the “effective amount”. The dosage schedule and effective amounts for this use, i.e., the “dosing regimen” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., Remington's; Egleton and Davis, Peptides 18:1431-1439, 1997; Langer, Science 249:1527-1533, 1990.

In accordance with these methods, the agents and/or pharmaceutical compositions defined in accordance with the present invention may be co-administered with one or more other agents. Reference herein to “co-administered” means simultaneous administration in the same formulation or in two different formulations via the same or different routes or sequential administration by the same or different routes. Reference herein to “sequential” administration is meant a time difference of from seconds, minutes, hours or days between the administration of the two types of agents and/or pharmaceutical compositions. Co-administration of the agents and/or pharmaceutical compositions may occur in any order.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is unacceptably toxic or if it would otherwise require too high a dosage or if it would not otherwise be able to enter the target cells.

Instead of administering the agents directly, they could be produced in the target cell, e.g. in a viral vector such as described above or in a cell based delivery system such as described in U.S. Pat. No. 5,550,050 and International Patent Publication Numbers WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application Number 0 425 731A and International Patent Publication Number WO 90/07936.

In yet another aspect, the present invention provides kits comprising the compositions e.g. agents of the present invention. The kits can also contain instructional material teaching the methodologies and uses of the invention, as described herein.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

The present invention is fuither described by the following non-limiting examples.

### EXAMPLE 1

### Experimental Procedures

The following experimental procedures are used in the subsequent Examples which follow.

Expression constructs:

Human Bcl-2 (Acc. no. NP_{—}000624; residues 1-217) and human Bcl-w (Acc. no. NP_{—}004041; residues 1-164; C29S A128E) were cloned into pQE-9 (Qiagen); expressed proteins have additional N-terminal residues (MRGSHHHHHHGS, SEQ ID NO:1). Human Bcl-x_{L} (Acc. no. NP_{—}612815; residues 1-209), mouse Mcl-1 (Acc. no. NP_{—}031588; residues 152-308) and mouse A1 (Acc. no. NP_{—}033872; residues 1-152) were cloned into pGEX-6P-3 (Amersham Biosciences) such that only five additional vector-derived residues (GPLGS) were present in the proteins following PreScission protease digestion (see below). FLAG (DYKDDDDK, SEQ ID NO:2)-tagged mammalian expression vectors for human Bcl-2, human Bcl-x_{L} and mouse Mcl-1 are described in Huang et al., EMBO J 16:4628-4638, 1997. N-terminally HA (YPYDVPDYA, SEQ ID NO:3)-tagged full-length human Bim_{EL}, human Bim_{L}, human Puma, mouse Bad, human Bik and mouse Noxa were sub-cloned into pEF PGKhygro (Huang et al., Supra; O'Conner et al., EMBO J 17:384-395, 1998). Proof-reading Pfu polymerase (Stratagene) was used for PCR and the constructs verified by automated sequencing. Details of oligonucleotides used and constructs are available from the inventors.

The constructs, in pQE-9 (Qiagen) include human (h) Bcl-2 (Acc. no. NP_{—}000624; residues 1-217), and hBcl-w (NP_{—}612815; residues 1-209) with C29S and A128E mutations to improve its stability (Hinds et al., 2003). The HexaHis tag (HHHHHH) allowed their purification on a nickel column. Recombinant hBcl-x_{L} ΔC24, mouse (m) Mcl-1 ΔN151 ΔC23 and mA1 ΔC20 were expressed as GST fusion proteins and cleaved from glutathione-sepharose columns with PreScission protease and purified as described (Day et al., 1999; Hinds et al., 2003). The constructs used, in pGEX-6P-3 (Amersham Biosciences), included sequences from Bcl-x_{L} (NP_{—}612815; residues 1-209), Mcl-1 (NP_{—}031588; residues 152-308), and A1 (NP_{—}033872; residues 1-152). After the protease digestion, they retain five N-terminal vector-derived residues (GPLGS).

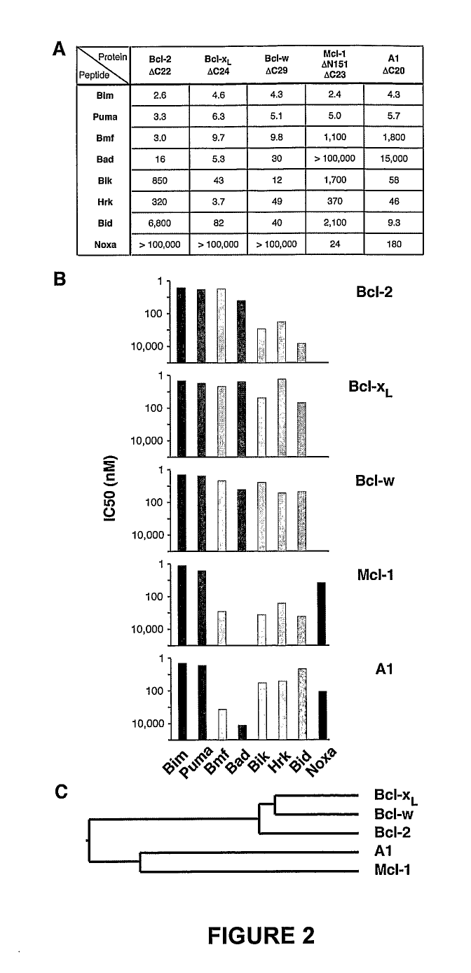

The peptides used in this study (FIG. 2A), which have free N- and C-termini, were synthesized by Mimotopes (Victoria, Australia). All peptides were purified by reverse-phase HPLC and were >90% pure, except for hbik (87%), mBmf (85%), and mNoxa B (78%). Their identities were confirmed by electrospray mass spectrometry. The peptides, quantified by weighing and by their absorbance at 214 nM on an analytical HPLC column, were completely dissolved as 1-2 mM stock solutions in water; hBimBH3 was dissolved in DMSO. The accession numbers on which the peptides were based are: mBim_{L} (AAC40030), hBim_{L} (AAC39594), hPuma (AAK39542), mBmf (AAK38747), hBad (NP_{—}004313), hBik (NP_{—}001188), hHrk (NP_{—}003797), hBid (NP_{—}001187), hNoxa (NP_{—}066950), mNoxa (NP_{—}067426).

Recombinant proteins and peptides:

Recombinant human Bcl-2 ΔC22 and human Bcl-w ΔC29 (containing C29S and A128E mutations to improve protein solubility but not affecting binding characteristics), expressed as N-terminal HexaHis fusion proteins, were prepared as described in Wilson-Annan et al., J. Cell Biol 162:877-888, 2003. Recombinant human Bcl-x_{L} ΔC24, mouse Mcl-1 ΔN151 ΔC23 and mouse A1 ΔC20 were expressed as GST fusion proteins and cleaved off Glutathione Sepharose columns as described (Day et al., Cell Death Differ 6:1125-1132, 1999; Hinds et al., EMBO J 22:1497-1507, 2003).

The peptides used in this study, with free N- and C-termini, were synthesized by Mimotopes. All peptides purified by reverse-phase HPLC were greater than 90% pure, except for Bik (87%), m Bmf (85%), and m Noxa2 (78%). Their identities were confirmed by electrospray Mass Spectrometric analyses. The peptides were quantified by weighing and by their absorbance at 214 nM on an analytical HPLC column; they were completely dissolved as 1-2 mM stock solutions in water. The accession numbers for the peptides were: mouse Bim_{L} (AAC40030), human BimL (AAC39594), human Puma (AAK39542), mouse Bmf (AAK38747), human Bad (NP_{—}004313), human Bik (NP_{—}001188), human Hrk (NP_{—}003797), human Bid (NP_{—}001187), human Noxa (NP_{—}066950), and mouse Noxa (NP_{—}067426).

Circular dichroism (CD) spectroscopy:

For the circular dichroism (CD) measurements, the stock solutions of peptides and horse heart myoglobin were diluted to a final concentration of 0.15 mg/ml in either 30 mM sodium phosphate (pH 7) or in 20 mM sodium phosphate (pH 7) supplemented with 30% (v/v) (2,2,2-Trifluoroethanol) (TFE). CD spectra were recorded at room temperature on an AVIV 62DS model spectropolarimeter with 0.1 cm cuvette. Two sequential scans were recorded and the background spectrum of the buffer alone was subtracted.

Affinity measurements and solution competition assays:

Affinity measurements were performed at room temperature on a Biacore 3000 biosensor (Biacore) with HBS (10 mM HEPES pH 7.2, 150 mM NaCl, 3.4 mM EDTA, 0.005% Tween 20) as the running buffer. Mouse 26-mer ^{wt}BimBH3, ^{4E}BimBH3 mutant, BadBH3, NoxaBH3 or control irrelevant peptides were immobilized onto CM5 sensor chips using amine-coupling chemistry (Wilson-Annan et al., Supra). To assess the binding affinities of pro-survival Bcl-2-like proteins for BimBH3 directly, the proteins were directly injected into the sensor chip at 20 μl/min. Residual bound proteins were desorbed with 50 mM NaOH or 6 M GuHCl (pH 7.2), followed by two washes with running buffer. Binding kinetics were derived from sensorgrams, following subtraction of baseline responses, using BIA evaluation software (version 3, Biacore) (Wilson-Annan et al., Supra).

The relative affinities of BH3 peptides for pro-survival Bcl-2 proteins were assessed by comparing their abilities to compete with immobilized ^{wt}BimBH3 peptide for binding to Bcl-2-like proteins (Wilson-Annan et al., Supra). A fixed sub-saturating amount (10 nM) of a pro-survival Bcl-2 protein was incubated with varying amounts of competitor BH3 peptide in HBS for >2 hr on ice. The mixtures were then injected over a sensor chip containing a channel bearing mouse ^{wt}BimBH3 and a control channel with mouse ^{4E}BimBH3 immobilized. The baseline response (control channel) was subtracted to obtain the absolute binding response. Taking the response for the pro-survival protein alone as the maximal response (100%), the relative residual binding (%) in the presence of increasing amounts of competitor peptides at a given injection time point (430 s) was then calculated. The relative residual responses (f) were plotted against initial peptide concentrations and fitted to the equation f=100/(1+(c/IC₅₀)^{m}), where c=concentration of competitor peptide, m=curvature constant, and IC₅₀=concentration of competitor peptide required to reduce binding by 50%. Theoretically, IC50=[A]/2+K_{D}, where [A] is the analyte concentration.

Some of recombinant proteins (Bcl-2, Bcl-x_{L}, A1) studied contain cysteine residues but the behavior of Bcl-2 or Bcl-x_{L} was not affected by dithiothreitol (DTT). 2.5 mM Tris-(carboxyethyl)phosphine hydrochloride (TCEP) was included in the incubation mixtures with A1, which contains two cysteines and appears to be less stable than the other proteins studied.

Transient transfection, immunoprecipatation and immunoblotting:

The maintenance, transfection and metabolic labeling of the human embryonic kidney (HEK) 293T cells with ³⁵S-methionine/cysteine (NEN) as well as co-immunoprecipitation have been described (Huang et al., Supra; Moriishi et al., Proc Natl Acad Sci USA 96:9683-9688, 1999; O'Conner et al., Supra). Briefly, equivalent TCA-precipitable lysates were immunoprecipiated using the mouse monoclonal antibodies to HA (HA.11; CRP), FLAG (M2; Sigma) and control Glu-Glu (CRP) tags. The proteins were resolved by SDS:PAGE, transferred onto nitrocellulose membranes and detected by fluorography (Amplify; Amersham Biosciences). Immunoblotting was performed using rat monoclonal antibodies to HA (3F10; Roche), FLAG (9H1; (Wilson-Annan et al., Supra) or mouse monoclonal anti-14-3-3β (H-8; Santa Cruz) detected by HRP-conjugated anti-rat (Southern Biotechnology) or anti-mouse (Silenus) antibodies. The proteins were revealed by enhanced chemiluminescence (ECL; Amersham Biosciences).

### EXAMPLE 2

### BimBH3 binds Bcl-2, Bcl-x_{L}, Bcl-w, Mcl-1 and A1 tightly

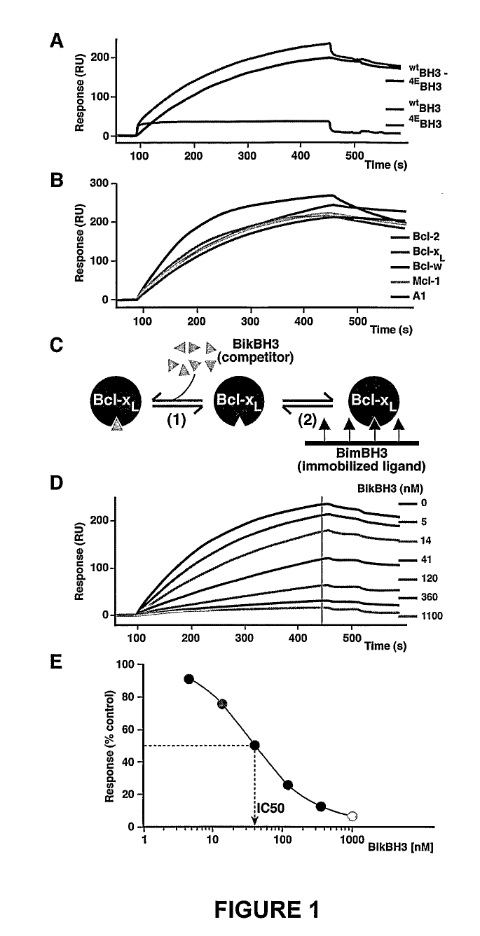

Generating the soluble, monomeric and equivalent recombinant pro-survival proteins needed for comparative in vitro binding studies required removal of their hydrophobic C-terminal domains (e.g. Hinds et al., Supra) and the N-terminal PEST region of Mcl-1 (Kozopas et al., Proc Natl Acad Sci 90:3516-3520, 1993). Likewise, full-length BH3-only proteins could not be reliably produced, so we used long peptides (24-26 residues; Table 3), because the reduced helical propensity of shorter ones can reduce binding (Petros et al., 2000, Supra). Since a 26-mer peptide spanning the BH3 domain of mouse Bim (BimBH3) binds to Bcl-w as avidly as longer Bim polypeptides (Wilson-Annan et al., Supra), we used it to measure the affinity of Bim for the other mammalian pro-survival molecules. Mcl-1, for example, gave a strong response when run over the immobilized wild-type but not over a mutant (non-binding) BimBH3 peptide (FIG. 1A). Indeed, Bcl-2, Bcl-x_{L}, Bcl-w, Mcl-1 and A1 all showed strong 1:1 stoichiometric binding to ^{wt}BimBH3 (FIG. 1B), the dissociation equilibrium constants (K_{D}) ranging from 0.2 to 4.5 nM (Table 2). Thus, Bim targets all five mammalian pro-survival proteins comparably.

TABLE 2
COMPARABLE BINDING OF BIM TO PRO-SURVIVAL PROTEINS

| Analyte | K_{D} (nM) | k_{d} (10⁻³ s⁻¹) | kₐ (10³ M⁻¹ s⁻¹) |
|---|---|---|---|
| Bcl-2 ΔC22 | 4.5 | 0.14 | 30 |
| Bcl-x_{L} ΔC24 | 0.8 | 0.44 | 570 |
| Bcl-w ΔC29 | 1.6 | 2.70 | 1700 |
| Mcl-1 ΔN151 ΔC23 | 0.2 | 0.26 | 1300 |
| A1 ΔC20 | 0.5 | 0.14 | 290 |

The binding constants of the pro-survival molecules for ^{wt}BimBH3, immobilized on sensor chip, were determined using biosensor experiments as described in Hinds et al., Supra; Wilson-Annan et al., Supra.

### EXAMPLE 3

### Certain BH3 domains bind pro-survival targets selectively

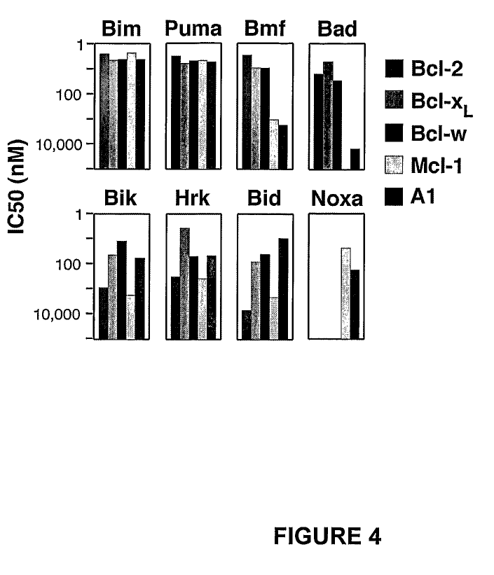

To assess if other BH3 peptides bind pro-survival proteins similarly, we directly compared their binding affinities in solution using a competitive binding assay. In such assays, the quality and absolute quantity of target proteins are less critical than in direct binding, and solution binding precludes the steric hindrance encountered with some immobilized peptides. FIG. 1C illustrates the approach: pre-incubation of Bcl-x_{L} in solution with increasing amounts of BikBH3 reduced its binding to ^{wt}BimBH3 (FIG. 1D). From the attenuation in binding, the IC50 (competitor peptide concentration that halves binding) of BikBH3 can be calculated (FIG. 1E). As the IC50 values reflect relative binding affinities (see Experimental Procedures), we used this assay to compare the binding affinities of eight BH3 peptides (Table 3) to the five pro-survival proteins. Strikingly, the relative affinities of the BH3 peptides for different pro-survival proteins varied over 10,000-fold (FIG. 2A and Table 2). Their binding properties fell into several classes (FIG. 2B and FIG. 4). Only BimBH3 and PumaBH3 had comparable affinities for all the pro-survival proteins. The other BH3 peptides were surprisingly selective for particular subsets of them. BadBH3, for example, strongly preferred Bcl-2, BCl-x_{L} and Bcl-w (5.3 to 30 nM) to A1 (−15 μM) or Mcl-1 (>100 μM) (FIGS. 2A and B), and BmfBH3 showed similar preferences. In marked contrast, NoxaBH3 was highly selective for Mcl-1 and A1 (nM range) but did not bind detectably to the others (>100 μM). Finally, BikBH3, HrkBH3 and BidBH3 preferred Bcl-x_{L}, Bcl-w and A1 over Bcl-2 or Mcl-1. Contrary to the prevailing view, the pro-survival proteins also had unique patterns for BH3 binding: Bcl-x_{L} behaved like Bcl-w, with Bcl-2 more distinct, whereas Mcl-1 and A1 formed a separate group (FIG. 2B).

TABLE 3
BH3 PEPTIDES

| | | | | | | | | | | | | | | | |
|---|---|---|---|---|---|---|---|---|---|---|---|---|---|---|---|
| | | | | | | | | H1 | | | | H2 | | | H3 |
| m ^{wt}BimBH3 (83-108) | D | L | R | P | E | I | R | I | A | Q | E | L | R | R | I |
| m ^{4E}BimBH3 (83-108) | — | — | — | — | — | — | — | E | — | — | — | E | — | — | E |
| Bim (81-106) | D | M | R | P | E | I | W | I | A | Q | E | L | R | R | I |
| Puma (130-155) | E | E | Q | W | A | R | E | I | G | A | Q | L | R | R | M |
| m Bmf (128-151) | | H | R | A | E | V | Q | I | A | R | K | L | Q | C | I |
| Bad (103-128) | N | L | W | A | A | Q | R | Y | G | R | E | L | R | R | M |
| Bik (51-75) | | M | E | G | S | D | A | L | A | L | R | L | A | C | I |
| Hrk (26-51) | R | S | S | A | A | Q | L | T | A | A | R | L | K | A | I |
| Bid (81-104) | | | D | I | I | R | N | I | A | R | H | L | A | Q | V |
| Noxa (18-43) | P | A | E | L | E | V | E | C | A | T | Q | L | R | R | F |
| m Noxa1 (16-41) | R | A | E | L | P | P | E | F | A | A | Q | L | R | K | I |
| m Noxa2 (68-93) | P | A | D | L | K | D | E | C | A | — | Q | L | R | R | I |
| | | | | | | | | H1 | | | | H2 | | | H3 |

| | | | | | | | | | | | | | |
|---|---|---|---|---|---|---|---|---|---|---|---|---|---|
| | | | | H4 | | | | | | | | | |
| m ^{wt}BimBH3 (83-108) | G | D | E | F | N | E | T | Y | T | R | R | | ^{wt}BimBH3 |
| m ^{4E}BimBH3 (83-108) | — | — | — | E | — | — | — | — | — | — | — | | ^{4E}BimBH3 |
| Bim (81-106) | G | D | E | F | N | A | Y | Y | A | R | R | | Bim |
| Puma (130-155) | A | D | D | L | N | A | Q | Y | E | R | R | | Puma |
| m Bmf (128-151) | A | D | Q | F | H | R | L | H | T | Q | | | m Bmf |
| Bad (103-128) | S | D | E | F | V | D | S | F | K | K | G | | Bad |
| Bik (51-75) | G | D | E | M | D | V | S | L | R | A | P | | Bik |
| Hrk (26-51) | G | D | E | L | H | Q | R | T | M | W | R | | Hrk |
| Bid (81-104) | G | D | S | M | D | R | S | I | P | P | G | | Bid |
| Noxa (18-43) | G | D | K | L | N | F | R | Q | K | L | L | | Noxa |
| m Noxa1 (16-41) | G | D | K | V | Y | C | T | W | S | A | P | | m Noxa1 |
| m Noxa2 (68-93) | G | D | K | V | N | L | R | Q | K | L | L | N | m Noxa2 |
| | | | | H4 | | | | | | | | | |

The immobilized peptides (top 2 rows) were derived from mouse (m) Bim_{L}. ^{4E}BimBH3 has four hydrophobic residues (H1-H4) critical for interacting with the pro-survival proteins (see text) mutated to glutamic acids (E). Competitor peptides were derived from human proteins except those denoted “m” (mouse). The sequences were aligned using the GCG “PILEUP” program as described in Huang and Strasser, Supra

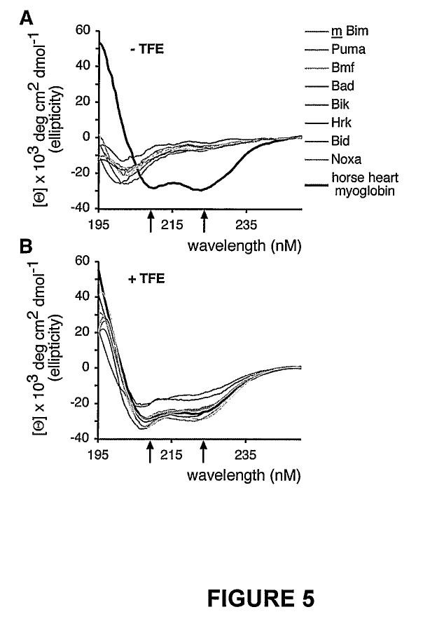

Since all the BH3 peptides bound avidly to at least two pro-survival proteins (FIG. 2), it is unlikely that their integrity or conformation affected our results. Nevertheless, as the binding of shorter BadBH3 peptides to Bcl-x_{L} depended on their helicity (Petros et al., 2000, Supra), we assessed the conformation of the peptides used by CD (circular dichroism) spectroscopy. In an aqueous environment, all the peptides appeared to be largely unstructured (FIG. 5A). However, on addition of 30% TFE (2,2,2-Trifluoroethanol), a solvent that stabilizes cc-helix formation (Nelson and Kallenbach, Biochemistry 28:5256-5261, 1989), they readily became α-helical (Supplemental FIG. S2B), indicating their helical potential. We observed no correlation between their helical propensity in TFE and binding affinities.

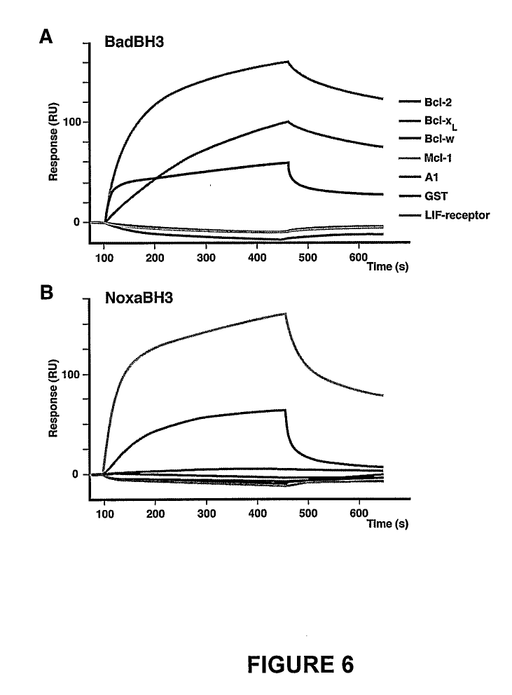

The distinctive and complementary binding patterns observed with Bad and Noxa were particularly striking (FIG. 2). The results with Noxa are consistent with those in another solution competition assay (Letai et al., Supra), but Noxa reportedly bound to Bcl-2 and Bcl-x_{L} in a co-immunoprecipitation assay (Oda et al., Science 288:1053-1058, 2000). To address this discrepancy, we also performed direct binding assays with immobilized BadBH3 or NoxaBH3 peptides. In accord with the solution competition results, only Bcl-2, Bcl-x_{L} and Bcl-w bound to BadBH3 (FIG. 6A) and only Mcl-1 and A1 to NoxaBH3 (FIG. 6B). Unlike human Noxa, mouse Noxa contains two BH3 domains (Oda et al., Supra), but both mouse Noxa BH3 peptides (Table 2) bound Mcl-1 (IC50 87 and 109 nM) but not Bcl-2, Bcl-x_{L} or Bcl-w. Therefore, Noxa presumably specifically antagonizes Mcl-1 and A1.

### EXAMPLE 4

### Bad, Bik and Noxa Have Selective Physiological Targets in Mammalian Cells

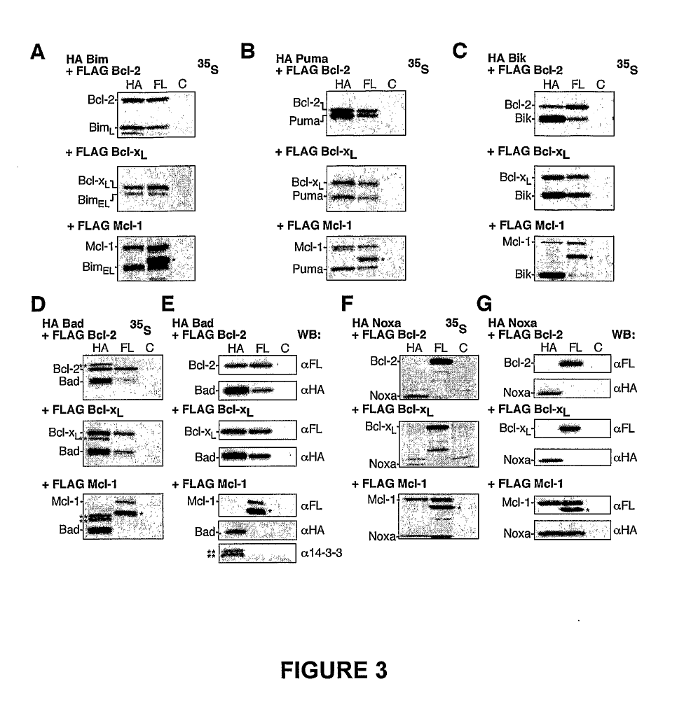

The selective interactions were confirmed in different assays. GST pull-down experiments, performed with the recombinant BH3-only proteins that could be made (Bim, Bmf, Bad, and Bid), gave results concordant with the solution competition assays. Most pertinently, GST Mcl-1 bound tightly to Bim, weakly to Bmf but not to Bad. To verify the in vitro findings, interactions of selected pairs of the full-length N-terminally tagged proteins were investigated in mammalian cells by co-immunoprecipitation. HA-Bim, Puma, Bad, Bik or Noxa were co-expressed in HEK293T cells with FLAG-Bcl-2, BCl-x_{L} and Mcl-1, as representatives of the three classes of pro-survival proteins profiled (FIG. 2B). The cells were metabolically labeled with ³⁵S-methionine/cysteine to permit semi-quantitative assessment of any interaction between co-associating radiolabeled proteins. For every pair tested, the interactions detected by co-immunoprecipitation (FIG. 3) concurred with the prior affinity measurements (FIG. 2). In particular, Bad bound well to Bcl-2 and Bcl-x_{L} but not appreciably to Mcl-1 (FIGS. 3D, 3E) (Opferman et al., Nature 426:671-b 676, 2003), whereas Noxa (full-length mouse Noxa bearing two BH3s) interacted only with Mcl-1 (FIGS. 3F, 3G). Bik bound Bcl-x_{L} to a greater extent than Bcl-2 or Mcl-1 (FIG. 3C), whereas Bim and Puma, as expected, bound all three pro-survival proteins well (FIGS. 3A and 3B).

### EXAMPLE 5

### Specific Residues Are Important for Determining Selectivity of Noxa for Mcl-1

When the sequences of BH3 domains were compared, it appears that that F32 and K35 may be important for determining the specificity of Noxa for Mcl-1. Wild-type Noxa did not bind Bcl-x_{L}, but mutation to either F32 or K35 enhances Bcl-x_{L} binding that is further enhanced in the double mutant (Table 4). This strongly suggests that F32 and K35 of Noxa are important for determining selectivity of Noxa for Mcl-1.

TABLE 4
SPECIFIC RESIDUES ARE IMPORTANT FOR DETERMINIG
SELECTIVITY OF Noxa FOR Mcl-1

| | | | | | | | | | | | | | | | | | | | | | | | | | | | IC50 |
|---|---|---|---|---|---|---|---|---|---|---|---|---|---|---|---|---|---|---|---|---|---|---|---|---|---|---|---|
| | | | | | | | | | | | | | | | | | | | | | | | | | | | for |
| | | | | | | | | | | | | | | | | | | | | | | | | | | | Bcl-x_{L} |
| | | | | | | | | | | | | | | | | | | | | | | | | | | | (nM) |
| Noxa | P | A | E | L | E | V | E | C | A | T | Q | L | R | R | F | G | D | K | L | N | F | R | Q | K | L | L | >100,000 |
| (18-43) | | | | | | | | | | | | | | | | | | | | | | | | | | | |
| Noxa | P | A | E | L | E | V | E | C | A | T | Q | L | R | R | F | G | D | E | L | N | F | R | Q | K | L | L | 5,800 |
| (18-43) mt1 | | | | | | | | | | | | | | | | | | | | | | | | | | | |
| Noxa | P | A | E | L | E | V | E | C | A | T | Q | L | R | R | I | G | D | K | L | N | F | R | Q | K | L | L | 1,100 |
| (18-43) mt2 | | | | | | | | | | | | | | | | | | | | | | | | | | | |
| Noxa | P | A | E | L | E | V | E | C | A | T | Q | L | R | R | I | G | D | E | L | N | F | R | Q | K | L | L | 110 |
| (18-43) mt3 | | | | | | | | | | | | | | | | | | | | | | | | | | | |
| | | | | | | | | | | | | | | | ↑ | | | ↑ | | | | | | | | | |

### EXAMPLE 6

### Bad, Bik and Noxa Have Distinct Targets in Mammalian Cells

To verify the in vitro findings, selected pairs of the full-length N-terminally tagged proteins associated in mammalian cells were investigated by co-immunoprecipitation. As representatives of the different classes of pro-survival proteins profiled (FIG. 4), Bcl-2, Bcl-x_{L} and Mcl-1 were co-expressed in 293T cells with Bim, Puma, Bik, Bad or Noxa. The cells were metabolically labeled with ³⁵S-methionine/cysteine to permit semi-quantitative assessment of any interaction between the labeled proteins or interaction was gauged by the more sensitive western blotting.

Significantly, for every pair tested, the interactions detected by co-immunoprecipitation were those expected from the prior affinity measurements (FIG. 4). In particular, Bad bound well to Bcl-2 and Bcl-x_{L} but not appreciably to Mcl-1 whereas Noxa (full-length mouse Noxa bearing two BH3s) interacted only with Mcl-1. Furthermore, Bik bound Bcl-x_{L} to a greater extent than Bcl-2 or Mcl-1, whereas Bim, as expected, bound avidly to all three pro-survival proteins, as did Puma.

### EXAMPLE 7

### BH3-only Proteins With Restricted Targets Are Weaker Killers

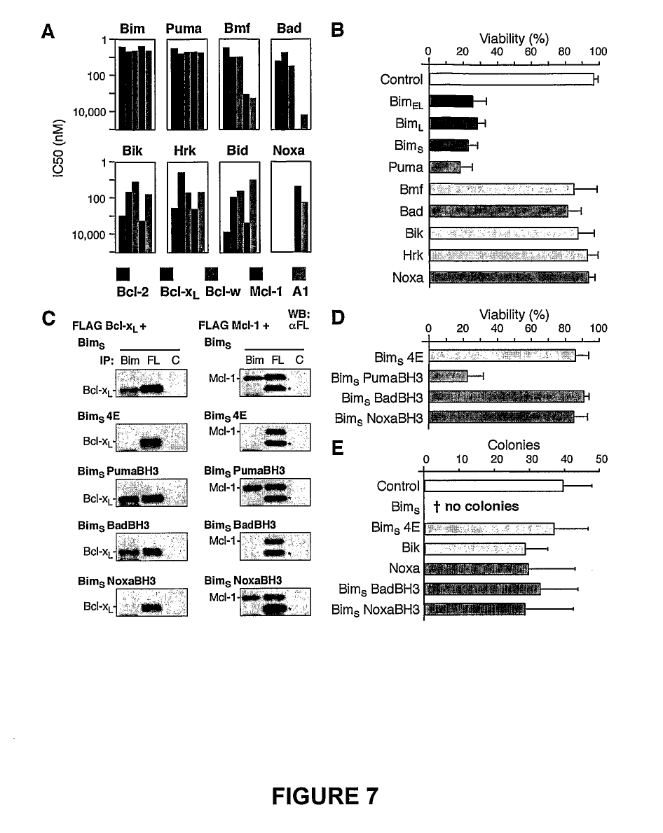

To assess the biological relevance of the selective binding, retroviral delivery was used to compare the ability of different BH3-only proteins to kill wild-type mouse embryonic fibroblasts (MEFs). To monitor expression of the introduced gene, a vector (pMIG) in which its expression is coupled via an internal ribosomal entry site (IRES) to that of green fluorescent protein (GFP) was used (Van Parijs et al., Immunity 11:281-288, 1999). The cells were efficiently infected (>90%) and the introduced BH3-only proteins were comparably expressed. Viability of the infected (GFP^{+ve}) cells was scored 24 hours after infection (FIGS. 7A, 7B) and in long-term colony assays (FIG. 7E).

Whereas infection with the parental virus caused minimal apoptosis, most cells infected with a virus expressing Puma or various Bim isoforms (Bimₛ, Bim_{L} or Bim_{EL}) soon died (FIG. 7B) in a dose-dependent manner and failed to generate colonies (FIG. 7E). Importantly, the other BH3-only proteins tested (Bmf, Bad, Bik, Hrk, Noxa), although adequately expressed were much less potent killers (FIG. 7B), probably because none of these BH3-only proteins can neutralize all the pro-survival molecules (Bcl-2, Bcl-x_{L}, Mcl-1) expressed substantially in MEFs.

The weaker pro-apoptotic activity of certain BH3 -only proteins, has usually been attributed to specific negative regulatory mechanisms, such as the binding of Bad by 14-3-3 proteins. To preclude such effects and allow direct comparison between different BH3 domains, chimeric molecules in which Bimₛ BH3 was replaced with that of Puma, Bad or Noxa were also analysed. Bimₛ was chosen as the common backbone because Bimₛ, the most potent Bim isoform is not regulated by interaction with the dynein motor complex. Its pro-apoptotic activity relies solely upon the BH3 region, because Bimₛ 4E, which has the BH3 mutated, does not bind any of the pro-survival molecules and lacks pro-apoptotic function. Another advantage of the chimeric proteins, unlike their native counterparts, is that all were expressed at comparable levels.

Notably, the Bimₛ chimera with the PumaBH3 retained Puma's ability to bind all the pro-survival molecules tested and killed as potently as native Bim or Puma. In contrast, the Bimₛ chimeras with the BadBH3 or the NoxaBH3 behaved like native Bad or Noxa respectively. Bimₛ BadBH3 bound Bcl-x_{L} but not Mcl-1, whereas Bimₛ NoxaBH3 instead bound Mcl-1. Accordingly, these two chimeras exhibited weak pro-apoptotic activities in both short- and long-term assays. Thus, the killing induced by each BH3-only protein appears to reflect its ability to bind tightly to all the pro-survival Bcl-2-like molecules present in the cell.

### EXAMPLE 8

### Functional Complementation Between Bad and Noxa

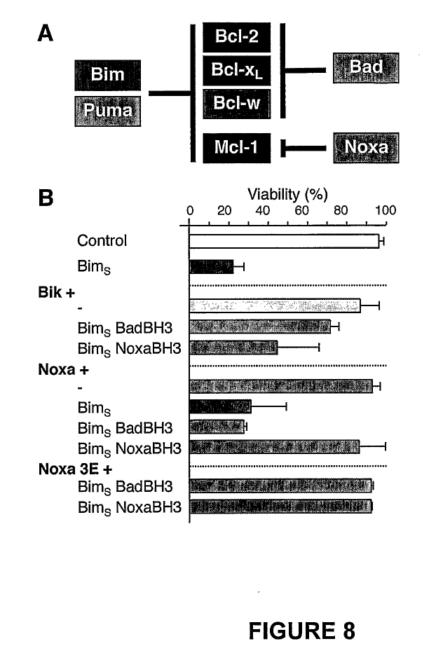

Since the results show that apoptosis requires neutralization of all the relevant pro-survival proteins, BH3-only proteins with complementary binding profiles (FIG. 8A) should cooperate in killing cells. Thus, the neutralization of Mcl-1 by Noxa should be complemented by co-expression of a BadBH3 to neutralize Bcl-2 and Bcl-x_{L} in MEFs (FIG. 8A). Likewise, the NoxaBH3 should augment killing induced by Bik, which binds to Mcl-1˜40-fold less well than to Bcl-x_{L}. To allow tests of complementation, pairs of BH3-only proteins were co-expressed in the pMIG vector by replacing GFP with GFP fusions of Bimₛ, or of the chimeric Bimₛ BadBH3 and Bimₛ NoxaBH3. Such fusions behave like their parental BH3-only counterparts, since a GFP Bimₛ fusion was a potent killer (FIG. 8B).

Importantly, co-expression of Noxa with the BadBH3 resulted in as much cell death as expression of Bimₛ alone (FIG. 8B). Similarly, the NoxaBH3 potently enhanced killing by Bik. The observed synergies reflect complementary BH3 function, because co-expression of a NoxaBH3 did not augment Noxa killing, and no significant killing (FIG. 8B) was observed when Bimₛ BadBH3 fused to GFP was co-expressed with an inert form of Noxa (the ³EBH3 mutant; see Experimental Procedures), which does not bind Mcl-1. Thus, both Bad and Bik, which predominantly bind Bcl-2 and Bcl-x_{L}, can potently cooperate with Noxa, which selectively binds Mcl-1, to augment the weak killing observed when any of the three BH3-only proteins is expressed alone in fibroblasts.

### EXAMPLE 9

### Potent Killing Induced by a Promiscuous Noxa Mutant

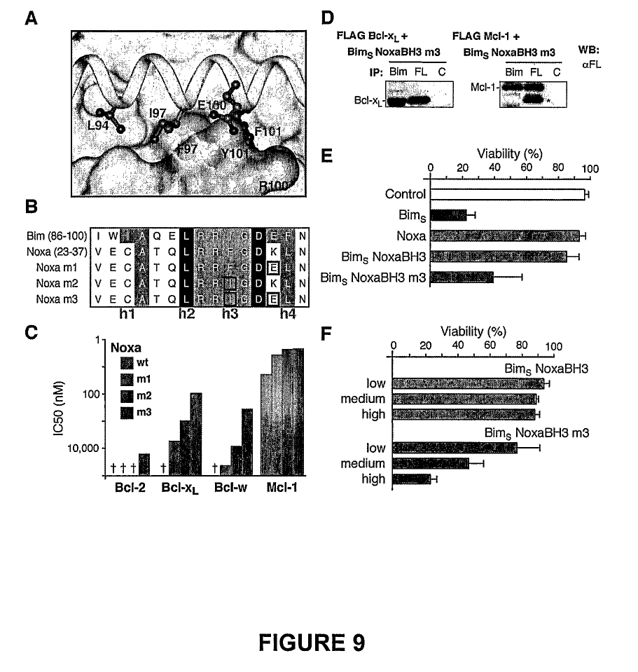

The molecular basis of the selective binding profile and weak killing activity of Noxa was then explored. The functional complementation results (FIG. 8) suggest that a form of Noxa that binds additional pro-survival proteins would be a potent killer. A high resolution X-ray crystallographic structure of the Bcl-x_{L}: Bim complex guided the search for such a variant (FIG. 9A). The hydrophobic residues of BimBH3 (including leucine L94, isoleucine I97, phenylalanine F101; based on mouse Bim_{L} numbering) bind hydrophobic pockets on the surface of Bcl-x_{L}. The pocket for Bim I97 (h3) on Bcl-x_{L} (and Bcl-2) is partly formed by phenylalanine (F97) and tyrosine (Y101) residues, which are larger than the corresponding residues in Mcl-1 (valine, histidine) or in A1 (valine, valine), suggesting that Mcl-1 and A1 can more readily accommodate the γ-branched phenylalanine (F32) found in the human NoxaBH3 (FIG. 9B). Furthermore, a mutual charge pair is formed between the negatively charged glutamate (E100) of Bim and positively charged arginine (R100) on Bcl-x_{L}, but this arginine, conserved in Bcl-2 and Bcl-w, is replaced in Mcl-1 by neutral asparagine and in A1 by a glutamate. Hence, the charge change from glutamate (E100) in the BimBH3 to lysine (K35) present in Noxa (FIG. 9B) may be a factor that impairs NoxaBH3 binding to Bcl-x_{L}.

Based on these considerations, Noxa peptides with the mutations K35E (m1), F32I (m2) or both together (m3) (FIG. 9B) were tested for binding to pro-survival proteins in solution competition assays. Their binding to Bcl-2 remained weak, but binding to Bcl-x_{L} and Bcl-w increased markedly (FIG. 9C). For Bcl-x_{L}, the charge switch mutation K35E (m1) increased binding more than 20 fold; the F32I substitution (m2) enhanced binding more than 100 fold; and the double mutation (m3) further augmented binding (FIG. 9C). Whereas the IC50 for wt NoxaBH3 was >100,000 nM for both Bcl-x_{L} and Bcl-w, the m3 mutant exhibited an IC50 of 110 nM for Bcl-x_{L} and 410 nM for Bcl-w, while Mcl-1 binding was slightly improved (FIG. 9C). Co-immunoprecipitation experiments confirmed that Noxa m3 bound Bcl-x_{L} in cells (FIG. 9D), whereas wild-type Noxa did not (FIG. 7C). Thus, the change of two key BH3 residues sufficed to broaden the specificity of Noxa.

Significantly, when expressed in fibroblasts, Noxa m3 proved to be a more potent killer than wild-type Noxa (FIGS. 9E, 9F). Thus, the binding of Noxa to Mcl-1 alone is not sufficient to kill MEFs. Efficient induction of apoptosis by Noxa requires additional BH3 interactions with proteins of the Bcl-2/Bcl-x_{L}/Bcl-w class, provided either by co-expression of Bad-like proteins (FIG. 8) or by mutations that decrease Noxa selectivity (m3; FIG. 9).

### BIBLIOGRAPHY

Adams et al., Nature 318:533-538, 1985
Adams et al., Genes Dev 17:2481-2495, 2003
Alving et al., Immunol Rev 145:5-31, 1995
Bleicher et al., Nat Rev Drug Discov 2:369-378, 2003
Cory et al., Nat Rev Cancer 2:647-656, 2002
Cory et al., Oncogene 22:8590-8607, 2003
Danial and Korsmeyer, Cell 116:205-219, 2004
Day et al., Cell Death Differ 6:1125-1132, 1999
Day et al., 1999; Hinds et al., 2003
Erickson et al., Science 249:527-533, 1990
Fix, Pharm Res 13:1760-1764, 1996
Glickman et al., J Biomol Screen 7:3-10, 2002
Hajduk et al., J Med Chem 42:2315-2317, 1999
Hinds et al., EMBO J 22:1497-1507, 2003
Huang and Strasser, Cell 103:839-842, 2000
Johnson et al., Peptide Turn Mimetics in Biotechnology and Pharmacy, Pezzuto et al., Eds., Chapman and Hall, New York, 1993
Kozopas et al., Proc Natl Acad Sci 90:3516-3520, 1993
Kuntz, Science 257:1078-1082, 1992
Langer, Science 249:1527-1533, 1990
Letai et al., Cancer Cell 2:183-192, 2002
Liu, X et al., Immunity 19:341-352, 2003
Lugovskoy et al., J Am Chem Soc 124:1234-1240, 2002
Moriishi et al., Proc Natl Acad Sci USA 96:9683-9688, 1999
Nelson and Kallenbach, Biochemistry 28:5256-5261, 1989
Opferman et al., Nature 426:671-676, 2003
Patton, Nat Biotech 16:141-143, 1998
Pellecchia et al., Nat Rev Drug Discov 1:211-219, 2002
Petros et al., 2000, Supra
Puthalakath et al., Cell Death Differ 9:505-512, 2002
Putney and Burke, Nat Biotech 16:153-157, 1998
Remington's; Akimaru et al., Cytokines Mol Ther 1:197-210, 1995
Remington's; Egleton and Davis, Peptides 18:1431-1439, 1997
Samanen et al., J Pharm Pharmacol 48:119-135, 1996
Saftler et al., 1997, Supra
Sayani and Chien, Crit Rev Ther Drug Carrier Syst 13:85-184, 1996
Strasser et al., Nature 348:331-333, 1990
Suntres and Shek, J Pharm Pharmacol 46:23-28, 1994
Szoka and Papahadjopoulos, Ann Rev Biophys Bioeng 9:467-508, 1980
van de Waterbeemd and Gifford, Nat Rev Drug Disc 2:192-204, 2003
Van Parijs et al., Immunity 11:281-288, 1999
Vaux et al., Nature 335:440-442, 1988
Vutla et al., J Pharm Sci 85:5-8, 1996
Wei et al., Science 292:727-730, 2001
Wells, Methods Enzymol 202:2699-2705, 1991
Wilson-Annan et al., J Cell Biol 162:877-888, 2003
Wohnsland et al., J Med Chem 44:923-930, 2001
Woodle et al., Pharm Res 9:260-265, 1992
Zhang, Nat Rev Drug Discov 1:101-102, 2002

1-18. (canceled)

19. A method for generating an antagonist of the pro-survival Bcl-2 family, said method comprising;
selecting a scaffold BH3-only protein structure with residue positions defining an amphipathic a-helix formed by the BH3 domain;
selecting one or more residue positions associated with a promiscuous binding phenotype of a BH3-only protein;
substituting amino acid residues conferring a promiscuous phenotype for a molecule selected from the group consisting of amino acids and amino acid chemical analogs which confer a restrictive binding pattern to a Bcl-2 protein; and
analyzing the interaction of each substitution for an ability to induce a more restrictive spectrum of binding to a Bcl-2 protein.

20. The method of claim 19 wherein said Bcl-2 antagonist is specific for one or more molecules selected from the group consisting of Bcl-2, Bcl-x_{L}, Bcl-w, Mcl and A1.

21. The method of claim 19 wherein said Bcl-2 antagonist is based on the structure of a molecule selected from the group consisting of Noxa, Bim, Puma, Bmf, Bad, Bik, Hrk and Bid.

22. The method of claim 19 wherein said Bcl-2 antagonist inhibits a pro-survival protein on a cancer cell.

23. The method of claim 22 wherein said cancer cell is selected from the group consisting of ABL 1 protooncogene, AIDS Related Cancers, Acoustic Neuroma, Acute Lymphocytic Leukaemia, Acute Myeloid Leukaemia, Adenocystic carcinoma, Adrenocortical Cancer, Agnogenic myeloid metaplasia, Alopecia, Alveolar soft-part sarcoma, Anal cancer, Angiosarcoma, Aplastic Anaemia, Astrocytoma, Ataxia-telangiectasia, Basal Cell Carcinoma (Skin), Bladder Cancer, Bone Cancers, Bowel cancer, Brain Stem Glioma, Brain and CNS Tumors, Breast Cancer, CNS Tumors, Carcinoid Tumors, Cervical Cancer, Childhood Brain Tumors, Childhood Cancer, Childhood Leukaemia, Childhood Soft Tissue Sarcoma, Chondrosarcoma, Choriocarcinoma, Chronic Lymphocytic Leukaemia, Chronic Myeloid Leukaemia, Colorectal Cancers, Cutaneous T-Cell Lymphoma, Dermatofibrosarcoma-protuberans, Desmoplastic-Small-Round-Cell-Tumor, Ductal Carcinoma, Endocrine Cancers, Endometrial Cancer, Ependymoma, Esophageal Cancer, Ewing's Sarcoma, Extra-Hepatic Bile Duct Cancer, Eye Cancer, Eye: Melanoma, Retinoblastoma, Fallopian Tube cancer, Fanconi Anaemia, Fibrosarcoma, Gall Bladder Cancer, Gastric Cancer, Gastrointestinal Cancers, Gastrointestinal-Carcinoid-Tumor, Genitourinary Cancers, Germ Cell Tumors, Gestational-Trophoblastic-Disease, Glioma, Gynaecological Cancers, Haematological Malignancies, Hairy Cell Leukaemia, Head and Neck Cancer, Hepatocellular Cancer, Hereditary Breast Cancer, Histiocytosis, Hodgkin's Disease, Human Papillomavirus, Hydatidiform mole, Hypercalcemia, Hypopharynx Cancer, IntraOcular Melanoma, Islet cell cancer, Kapos's sarcoma, Kidney Cancer, Langerhan's-Cell-Histiocytosis, Laryngeal Cancer, Leiomyosarcoma, Leukaemia, Li-Fraumeni Syndrome, Lip Cancer, Liposarcoma, Liver Cancer, Lung Cancer, Lymphedema, Lymphoma, Hodgkin's Lymphoma, Non-Hodgkin's Lymphoma, Male Breast Cancer, Malignant-Rhabdoid-Tumor-of-Kidney, Medulloblastoma, Melanoma, Merkel Cell Cancer, Mesothelioma, Metastatic Cancer, Mouth Cancer, Multiple Endocrine Neoplasia, Mycosis Fungoides, Myelodysplastic Syndromes, Myeloma, Myeloproliferative Disorders, Nasal Cancer, Nasopharyngeal Cancer, Nephroblastoma, Neuroblastoma, Neurofibromatosis, Nijmegen Breakage Syndrome, Non-Melanoma Skin Cancer, Non-Small-Cell-Lung-Cancer-(NSCLC), Ocular Cancers, Oesophageal Cancer, Oral cavity Cancer, Oropharynx Cancer, Osteosarcoma, Ostomy Ovarian Cancer, Pancreas Cancer, Paranasal Cancer, Parathyroid Cancer, Parotid Gland Cancer, Penile Cancer, Peripheral-Neuroectodermal-Tumors, Pituitary Cancer, Polycythemia vera, Prostate Cancer, Rare-cancers-and-associated-disorders, Renal Cell Carcinoma, Retinoblastoma, Rhabdomyosarcoma, Rothmund-Thomson Syndrome, Salivary Gland Cancer, Sarcoma, Schwannoma, Sezary syndrome, Skin Cancer, Small Cell Lung Cancer (SCLC), Small Intestine Cancer, Soft Tissue Sarcoma, Spinal Cord Tumors, Squamous-Cell-Carcinoma-(skin), Stomach Cancer, Synovial sarcoma, Testicular Cancer, Thymus Cancer, Thyroid Cancer, Transitional-Cell-Cancer-(bladder), Transitional-Cell-Cancer-(renal-pelvis-/-ureter), Trophoblastic Cancer, Urethral Cancer, Urinary System Cancer, Uroplakins, Uterine sarcoma, Uterus Cancer, Vaginal Cancer, Vulva Cancer, Waldenstrom's-Macroglobulinemia, and Wilms'Tumor.

24. The method of claim 19 wherein said Bcl-2 antagonist inhibits a prosurvival protein in a mammal.

25. The method of claim 24, wherein said mammal is a human.

26. A method for generating an antagonist of the pro-survival Bcl-2 protein family, said method comprising;
selecting a restrictive BH3-only protein as a scaffold protein;
determining the conformation of the scaffold conferring the restrictive phenotype; and
generating a chemical compound which mimics said scaffold and conformational part conferring a restrictive spectrum of binding to a Bcl-2 protein.

27. A method for selecting an antagonist of the pro-survival Bcl-2 protein family, said method comprising;
selecting a restrictive BH3-only protein as a scaffold protein;
determining the conformation of the scaffold conferring the restrictive phenotype; and
generating a chemical compound which mimics said scaffold and conformational part conferring a restrictive spectrum of binding to a Bcl-2 protein.

28. A computational method for designing an antagonist of the pro-survival Bcl-2 protein family based on a scaffold BH3-only protein with residue positions conferring a restrictive phenotype comprising;
selecting a collection of promiscuous BH3- only proteins;
providing a sequence alignment of said proteins and comparing said sequence alignment to a restrictive BH3-only protein;
generating a frequency of occurrence for individual amino acids in one or a plurality of positions with said alignments conferring promiscuity or restrictivity with respect to binding to Bcl-2 proteins;
creating a scoring function selected from the group consisting of charge, size, conformation, solubility, polarity, hydrophobicity, hydrophilicity and contribution to tertiary structure using said frequencies;
using said scoring function and at least one additional scoring function to generate information selected from the group consisting of a set of optimized protein sequences and the conformational equivalents of optimized protein sequences; and
performing an activity selected from the group consisting of generating a compound having a restrictive binding phenotype to a Bcl-2 protein and selecting a protein having a restrictive binding phenotype to a Bcl-2 protein.

29. The method of claim 28 wherein said scoring function is selected from the group consisting of the number and position of acidic residues, the number and position of basic residues, the number and position of polar residues, the number and position of non-polar residues, the number and position of charged residues, the number and position of uncharged residues, the number and position of hydrophilic residues, the number and position of hydrophobic residues, the levels of residues, the solubility levels of residues, the size of residues, and the contribution to tertiary structure the residue makes in the BH3-only protein.

30. A computer program product for determining the structure of an agent to induce apoptosis in a cell, said product comprising:
(1) code that receives as input scoring function (SF) for at least two features associated with a molecule selected from the group consisting of BH3-only and Bcl-2, wherein said features are selected from the group consisting of: (m) the number and position of acidic residues, (n) the number and position of basic residues, (o) the number and position of polar residues, (p) the number and position of non-polar residues, (q) the number and position of charged residues, (r) the number and position of uncharged residues, (s) the number and position of hydrophillic residues, (t) the number and position of hydrophobic residues, (u) the levels of residues, (v) the solubility levels of residues, (w) the size of residues, and (x) the contribution to tertiary structure the residue makes in the BH3-only protein;
(2) code that adds said SF to provide a sum corresponding to a Pv for BH3-only proteins; and
(3) a computer readable medium that stores the codes.

31. An apparatus for assessing the likely usefulness of a BH3-only protein or chemical equivalent to induce apoptosis in a cell comprising;
(1) a machine-readable data storage medium comprising a data storage material encoded with machine-readable data, wherein said machine-readable data comprise lys for at least two features associated with a molecule selected from the group consisting of BH3-only and Bcl-2, wherein said features are selected from the group consisting of: (m) the number and position of acidic residues, (n) the number and position of basic residues, (o) the number and position of polar residues, (p) the number and position of non-polar residues, (q) the number and position of charged residues, (r) the number and position of uncharged residues, (s) the number and position of hydrophillic residues, (t) the number and position of hydrophobic residues, (u) the levels of residues, (v) the solubility levels of residues, (w) the size of residues, and (x) the contribution to tertiary structure the residue makes in the BH3-only protein;
(2) a working memory for storing instructions for processing said machine-readable data;
(3) a central-processing unit coupled to said working memory and to said machine-readable data storage medium, for processing said machine readable data to provide a sum of said SF corresponding to a Pv for said compound (s); and
(4) an output hardware coupled to said central processing unit, for receiving said Pv.

32. A method of treating cancer in a subject comprising;
administering to said subject an effective amount of an antagonist of a Bcl-2 protein for a time and under conditions sufficient to decrease said cancer.

33. The method of claim 32, further comprising one or more pharmaceutically acceptable carriers or diluents.

34. The method of claim 32 wherein administration of said antagonist is accomplished by a method selected from the group consisting of respiratoral, intratracheal, nasopharyngeal, intravenous, intraperitoneal, subcutaneous, intracranial, intradermal, intramuscular, intraoccular, intrathecal, intracereberal, intranasal, infusion, oral, rectal, patch and implant rates.

35. A method of preventing cancer in a subject comprising;
administering to said subject an effective amount of an antagonist of a Bcl-2 protein for a time and under conditions sufficient to prevent said cancer.

36. The method of claim 35 further comprising one or more pharmaceutically acceptable carriers or diluents.

37. The method of claim 35 wherein administration of said antagonist is accomplished by a method selected from the group consisting of respiratoral, intratracheal, nasopharyngeal, intravenous, intraperitoneal, subcutaneous, intracranial, intradermal, intramuscular, intraoccular, intrathecal, intracereberal, intranasal, infusion, oral, rectal, patch and implant rates.

38. A composition comprising;
(a) an antagonist of the pro-survival Bcl-2 protein family said antagonist generated by the method of a scaffold BH3-only protein structure with residue positions defining an amphipathic α-helix formed by the BH3 domain;
(b) selecting one or more residue positions associated with a promiscuous binding phenotype of a BH3-only protein;
(c) substituting amino acid residues conferring a promiscuous phenotype for amino acids or their chemical analogs which confer a restrictive binding pattern to a Bcl-2 protein; and
(d) analyzing the interaction of each substitution for an ability to induce a more restrictive spectrum of binding to a Bcl-2 protein, said composition further comprising one or more pharmaceutically acceptable carriers and/or diluents.


SEQUENCE LISTING (3 sequences)

SEQ ID NO: 1 - protein, 12 aa, peptides
MRGSHHHHHHGS

SEQ ID NO: 2 - protein, 8 aa, peptides
DYKDDDDK

SEQ ID NO: 3 - protein, 9 aa, peptides
YPYDVPDYA